SEQUENCE LISTING (2 sequences)

SEQ ID NO: 1 - DNA, 26 bp, Artificial Sequence
  Synthetic Primer
ctggaaaatgaaataccggaggttga

SEQ ID NO: 2 - DNA, 26 bp, Artificial Sequence
  Syhnthetic Primer
cgtaatgtggcgcttgagcatgtaac

(19) United States
(12) Patent Application Publication
(10) Pub. No.: US 2014/0363819 A1
(43) Pub. Date: Dec. 11, 2014

(54) COMPOSITION TO OVERCOME INHIBITORS IN PCR AND GROWTH CULTURES

(71) Applicants: Katherine Rowlyk, St. Louis, MO (US); Zhian Zhang, Ballwin, MO (US); Milko Kermekchiev, St. Louis, MO (US)

(72) Inventors: Katherine Rowlyk, St. Louis, MO (US); Zhian Zhang, Ballwin, MO (US); Milko Kermekchiev, St. Louis, MO (US)

(21) Appl. No.: 13/997,194

(22) PCT Filed: Dec. 22, 2011
(86) PCT No.: PCT/US11/67040

§ 371 Date: Jul. 22, 2014

Related U.S. Application Data
(60) Provisional application No. 61/425,913, filed on Dec. 22, 2010.

(51) Int. Cl. C12Q 1/68; C12N 1/20; C12N 1/38

(52) U.S. Cl. CPC C12Q 1/689; C12N 1/38; C12Q 1/686; C12N 1/20
USPC 435/6.12; 435/244

(57) ABSTRACT

Provided herein are compositions and methods for improving amplification or detection of a target nucleic acid in a sample containing PCR inhibitors, such as polyphenols. One aspect provides an enhancer composition including casein or polyvinylpyrrolidone, or a modified polymer thereof. Another aspect provides a method of amplifying a target nucleic acid with an enhancer composition including casein or polyvinylpyrrolidone, or a modified polymer thereof. Another aspect provides a method for culturing microorganisms in media containing PVP or casein.

### CROSS-REFERENCE TO RELATED APPLICATIONS

The present application claims the benefit of U.S. Provisional Application Ser. No. 61/425,913 filed Dec. 22, 2010, which is incorporated herein by reference in its entirety.

### STATEMENT REGARDING FEDERALLY SPONSORED RESEARCH OR DEVELOPMENT

Not Applicable.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present disclosure generally relates to enhancement of nucleotide amplification reactions, such as conventional PCR, RT-PCR, real-time PCR, real-time RT PCR, and sequencing, especially, for samples containing polyphenols and other PCR inhibitors.

### BACKGROUND OF THE INVENTION

Common food-borne pathogens, such as Salmonella, are responsible for thousands of deaths in the U.S. every year. According to a 1999 study by the Centers for Disease Control and Prevention, food-borne pathogens cause around 76 million illnesses, 325,000 hospitalizations, and 5,000 deaths in the United States each year. Three pathogens are responsible for 1,500 of these deaths, Salmonella, Listeria, and Toxoplasma (Hedberg C.) Salmonella is gram-negative bacillus found in meat, eggs, dairy products, chocolate, seafood, and have been, and could again be used for domestic terrorism.

Rapid detection is desired for quick recalls of infected food products before they become available to consumers, as well as clinical diagnoses and other responses to an outbreak.

Traditional detection methods for food-borne pathogens take several days, as they rely on cell culture, isolation, and identification. Due to the long incubations required, it is preferable to use a rapid detection method and then verify positive results by traditional methods. For these rapid assays, accuracy, speed, labor, and cost are important factors. Typically, a relatively long cultural enrichment step (approximately 24 hours) is required, followed by shorter steps of sample preparation and detection (2-6 hours). However, in some cases pathogen detection from non-enriched samples may be possible, using concentration and capture techniques. This is desirable because it allows for much more rapid detection, and can also provide information about pathogen concentration which is lost during cultural enrichment. The most common rapid detection methods are antibody-based (ELISA, latex agglutination, and others), but nucleic-acid based methods are also popular. The most common nucleic-acid based method is PCR, but other methods are also in use, such as probes for rRNA (e.g. GeneQuence, Neogen).

PCR, with its ability to quickly amplify target DNA millions of times, has exceptional sensitivity and quick detection times, typically within 2-3 hours after cultural enrichment. The theoretical sensitivity of PCR for a single-copy gene, with the addition of 10 μl of sample into a 100 μl reaction, is 100 CFU/ml which is similar to traditional plating methods. However, in practice, sensitivity is much lower. Perhaps only ten fold lower in some foods, but inhibitory foods typically reduce sensitivity by 1000 fold or more. See review by Wilson I G.

PCR from non-enriched food samples results in the most rapid detection possible, as the cultural enrichment step is the longest time constraint on detection. But typical detection limits of PCR from non-enriched samples are very high, in the range of 10³-10⁸ CFU/g (Wilson I G.) Therefore, intensive sample processing/preparation is required, which can be laborious and costly.

There are several known mechanisms of PCR inhibition, including interference with cell lysis, degradation or sequestration of DNA, or direct interference with the DNA polymerase (Wilson I G.) Some inhibitors may also have a quenching effect on the dyes used for real-time PCR detection, as we noted with blood (Kermekchiev M B, et al.) Inhibitors affecting the DNA polymerase may do so by proteinase degradation of the enzyme, binding to the active site, or otherwise disturbing favorable reaction conditions (ionic strength, solute concentrations, etc.)

There are known inhibitors of PCR in food, culture media, and reagents of DNA extraction (Feng P., 2001, Rossen L, et al., Rådström P, et al., Wilson I G.). These inhibitors may compromise PCR and lead to potentially deadly false-negatives. An overnight cultural enrichment step is typically required for growth of the target pathogen and dilution of the inhibitors in food. With this enrichment step, PCR can usually achieve the federally-required detection limits of 0.04 CFU/g (1 Colony Forming Unit per 25 g food sample). In particularly troublesome foods, however, false negatives may still occur.

Many DNA polymerases are inhibited by polyphenolic compounds and other substances found in chocolate. Conventional DNA polymerase enzymes are inhibited at about 2 μg of crude chocolate per 50 μL reaction volume.

In cases when a component of the food or culture media is known to be inhibitory to PCR, the most common strategy is to dilute the inhibitors out. But this practice can dilute the target DNA and decrease the sensitivity of the assay.

Chocolate is reported to inhibit Salmonella growth in culture (Busta F F and Speck M L.) as well as PCR itself. The main inhibitory substances in chocolate are flavonoids, secondary plant metabolites also found in foods such as tea, wine, and fruits. Their polyphenolic structure allows flavonoids to interact with proteins, including the DNA polymerase, leading to inhibition of PCR (Shinozuka K et al.) In addition, there are known PCR inhibitors in the culture media standard for chocolate. Every year, there are dozens of recalls of chocolate products, in connection with Salmonella infected peanuts, due to the common combination of chocolate and peanut butter. Because the inhibition of PCR by chocolate substantially slows down detection, a reliable rapid test for potentially infected products would decrease future outbreaks.

Various additives to PCR have been reported to relieve inhibition, such as organic solvents (DMSO), non-ionic detergents (Triton X and Tween20), solutes (betaine and glycerol), polymers (PEG 400), proteins (bovine serum albumin (BSA), bacteriophage T4's single-stranded DNA-binding protein gp32), or cocktails of protease inhibitors (Rådström P, et al.).

The pre-enrichment broth for detection of Salmonella in chocolate, adopted official first action by the Association of Official Analytical Chemists (AOAC), is nonfat dry milk with brilliant green dye (NFDM-BG). Chocolate tends to inhibit the growth of Salmonella (Busta F F and Speck M L), but this effect is reported to be alleviated somewhat by the presence of casein, the predominant protein found in milk (Zapatka F A, et al.). Milk proteinases are known inhibitors of PCR. (Powell H A, et al.).

Casein (an abundant milk protein) has been reported to alleviate the inhibitory effect of chocolate on bacterial survival and proliferation and allow culture thereof (Zapatka and Varney 1977 J. of Applied Bacter. 42, 21-25). Casein has been used in a PCR reaction mixture with bile salts and mucins at a concentration of 0.01% (Al-Soud et al. 2004. FEMS Immunology and Medical Microbiology 44, 177-182). Polyphenol astringency mediation by casein has been described (Luck et. al. 1994. Phytochemistry 37(2), 357-371).

Polyvinylpyrrolidone (PVP) has been reported to be effective at relatively low concentrations (e.g., less than 2%) for DNA extraction protocols for PCR (Home et al. 2004 Plant Molecular Biology Reporter 22, 83a-83i). PVP at a concentration of 1% to 2% has also been reported to reduce polyphenolic inhibition in PCR reactions, but higher or lower concentrations were observed to not be effective.

### SUMMARY OF THE INVENTION

Among the various aspects of the present disclosure is the provision of an enhancer composition including casein or a PVP polymer that can reduce or eliminate inhibitory effects on nucleic acid amplification reactions from chocolate or other PCR-inhibitor-containing samples, such as a polyphenol-containing sample.

One aspect provides a amplifying a target nucleic acid with a polymerase chain reaction (PCR) in a sample comprising a PCR inhibitory substance. In some embodiments, the method includes forming an assay mixture including a sample containing a target nucleic acid and a PCR inhibitory substance, such as polyphenols; at least one polymerase; and an enhancer composition that includes at least one of (i) casein or (ii) polyvinylpyrrolidone (PVP), or a modified polymer of PVP in an amount effective to reduce or eliminate inhibitory effects of a PCR inhibitory substance(s) of the sample; and amplifying the target nucleic acid in the assay mixture.

In some embodiments, the sample includes the PCR inhibitory substance. In some embodiments, the PCR inhibitory substance is a component of the PCR other than the sample. In some embodiments, the inhibitory substance is or includes a polyphenol.

In some embodiments, the enhancer composition comprises a casein selected from the group consisting of an αS1 casein, an αS2 casein, a β casein, a κ casein, or a paracasein. In some embodiments, the enhancer composition comprises casein a concentration of at least about 0.05%. In some embodiments, enhancer composition comprises casein a concentration of at least about 0.05% up to about 2.5%. In some embodiments, the enhancer composition comprises casein a concentration of about 0.4%.

In some embodiments, the enhancer composition comprises PVP or polyvinylpolypyrrolidone (PVPP). In some embodiments, the enhancer composition comprises PVP or PVPP at a concentration of at least about 0.1%. In some embodiments, the enhancer composition comprises PVP or PVPP at a concentration of at least about 0.1% up to about 25%. In some embodiments, the enhancer composition comprises PVP at a concentration of about 8% to about 10%. In some embodiments, the enhancer composition comprises PVPP at a concentration of about 1% to about 5%.

In some embodiments, the enhancer composition comprises at least one of trehalose, carnitine, a nonionic detergent, or heparin. In some embodiments, the enhancer composition comprises about 0.1 to about 0.8 M trehalose, about 0.1 M to about 1.5 M L-carnitine, or about 0.01 to about 8% nonionic detergent.

In some embodiments, the enhancer composition is a PEC enhancer composition with PVP, PVPP, or casein included in amounts as described above.

In some embodiments, the sample is an enriched sample. In some embodiments, the sample is an enriched sample; the enhancer composition comprises (i) casein or PVP, or a modification thereof, and (ii) (a) at least one of trehalose, carnitine, or a nonionic detergent; or (b) about 0.1 to about 0.8 M trehalose, about 0.1 M to about 1.5 M L-carnitine, or about 0.01 to about 8% nonionic detergent.

In some embodiments, the sample includes a plant material, blood or blood component, bile, dye, or soil. In some embodiments, the sample includes chocolate, potato skins, tea, berries, beer, wine, olive oil, walnuts, peanuts, tobacco, tomato, soybean, indigo dye, bile tannin, whole blood, blood serum, blood plasma, or soil.

In some embodiments, the sample comprises chocolate at a concentration of about 0.05 μg/μl up to about 20 μg/μl.

In some embodiments, the sample includes soil or soil extract at a concentration of at least about 1% up to about 90% of a total volume of the assay mixture or a soil or soil extract equivalent amount that provides up to about 25 ng of humic acid per 50 μL reaction volume.

In some embodiments, the sample includes a plant material or plant extract at a concentration of at least about 1% up to about 90% of a total volume of the assay mixture or a soil or soil extract equivalent amount that provides up to about 300 ng of polyphenols per 25 μl reaction volume.

In some embodiments, the sample includes a blood or a blood component at a concentration of at least about 1% up to about 25% of a total volume of the assay mixture.

In some embodiments, the target nucleic acid comprises a DNA or an RNA molecule. In some embodiments, the target nucleic acid comprises a pathogen DNA or an RNA molecule. In some embodiments, the target nucleic acid comprises a Salmonella DNA or an RNA molecule.

In some embodiments, the PCR is a reverse-transcriptase (RT) PCR or a real-time RT-PCR; the target nucleic acid comprises an RNA molecule; and the assay mixture further comprises a reverse-transcriptase.

In some embodiments, the assay mixture comprises at least one dye. In some embodiments, the at least one dye is selected from the group consisting of SYBR Green, Ethidium Bromide, PICO, TOTO, YOYO or LC Green. In some embodiments, the dye is present in the assay mixture at least about 0.5× up to about 50×, up to about 40×, up to about 30×, up to about 20×, or up to about 10×, where X is a manufacturer unit for concentration.

In some embodiments, the assay mixture comprises at least one DNA polymerase. In some embodiments, the at least one polymerase is selected from the group consisting of OmniTaq, Omni Klentaq, Omni Klentaq-LA, wild type Tag; FastStart Taq; JumpStart Taq; HotStart Plus Taq; AmpliTaq Gold, KlenTaq, FL-12, FL-10, and KT-12. In some embodiments, the assay mixture comprises at least two polymerases.

Another aspect provides an ecomposition for enhancing nucleic acid amplification in a sample comprising a PCR inhibitory substance. In some embodiments, the composition comprises (i) casein at a concentration of at least about 0.05% up to about 2.5%; or PVP or PVPP at a concentration of at least about 0.1% up to about 25%; and (ii) about 0.1 to about 0.8 M trehalose, about 0.1 M to about 1.5 M L-carnitine, or about 0.01 to about 8% nonionic detergent. In some embodiments, the composition is an effective PCR enhancer for samples containing polyphenols in an amount effective to inhibit PCR.

Another aspect provides a method of culturing a microorganism. In some embodiments, the method includes culturing a microorganism in a culture media, the culture media comprising PVP or Casein, or a modification thereof. In some embodiments, the PVP or casein is present in an amount effective to decrease, substantially eliminate, or eliminate growth inhibition effects of a substance in the culture media or increase or substantially increase microorganism growth rate. In some embodiments, the microorganism comprises Salmonella.

In some embodiments, the culture media includes a solid culture media, the solid culture media comprising PVP, or a modification thereof, in an amount of at least about 0% up to about 25%. In some embodiments, the culture media includes a solid culture media comprising PVP at least about 0% up to about 25% or Casein 0% up to 10%. In some embodiments, the culture media is a liquid culture media comprising PVP at least about 0% up to about 4% or Casein at least about 0% up to 10%. In some embodiments, a concentration of PVP in the culture media of greater than 4% inhibits microorganism growth. In some embodiments, the microorganism is Salmonella.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a series of agarose gel images showing chocolate tolerance of Taq, Klentaq 1, Klentaq 10, and FLAC2. Chocolate was added in increasing concentrations at 1:256 (“1”), 1:128 (“2”), 1:64 (“3”) dilution of a ˜1 g/ml solution of chocolate chips in water. The “0” reaction contained no chocolate. Further information regarding methodology is presented in Example 2.

FIG. 2 is a series of graphs showing chocolate tolerance of Taq and FLAC2 in real-time PCR. Further information regarding methodology is presented in Example 2.

FIG. 3 is series of agarose gel images showing PCT amplification of samples having decreasing amounts of enriched cocoa (20, 10, 5, and 2.5 μg per 50 μl reaction). PCR reactions contained OmniTaq polymerase (FIG. 3A) or Omni Klentaq polymerase (FIG. 3B) and either 8% PVP or no PVP. The lowest dilution was 2.5 ug chocolate per reaction. Further information regarding methodology is presented in Example 3.

FIG. 4 is a series of agarose gel images showing detection of Salmonella DNA in chocolate samples containing 200 μg of chocolate (equivalent to 2 μl primary enrichment) with 8% PVP (FIG. 4A) and without enhancer (FIG. 4B). Further information regarding methodology is presented in Example 3.

FIG. 5 is a series of images of chocolate samples containing Salmonella in solid and liquid growth media. FIG. 5A is an image of a solid media chocolate samples containing Salmonella plated on Hektoen Enteric (HE) Agar containing 0%, 5%, or 25% PVP. FIG. 5B is an image of liquid media chocolate samples containing Salmonella. Further information regarding methodology is presented in Example 4.

FIG. 6 is a series of images of gels showing five endogenous human gene targets amplified from crude samples containing 20% human whole blood with 0.5 μl Omni Klentaq per 50 μl reaction volume in the presence of increasing amounts of polyvinylpyrrolidone (PVP). One of the targets (2 kb globin gene) was also amplified in parallel reactions from 10 ng purified human DNA. The amplification was performed for 40 cycles using a 6 min, 95 degree initial heating step, and 3-6 minutes extension time. Only the PVP additives were used. The amplified products were analyzed in 1.5% ethidium bromide stained agarose gel. Lanes M, 100 bp DNA ladder.

FIG. 7 is an image of a gel showing PCR amplification of a Salmonella gene target from chocolate samples. A 783 bp Salmonella gene target was amplified in 50 μl PCR reactions containing 200 μg of chocolate using OmniTaq and varying percentages of PVP, with and without the PCR Enhancer Cocktail (PEC).

FIG. 8 is an image of a gel showing synergy of PEC and PVP on PCR in chocolate samples. Different concentrations of chocolate were added to 50 μl PCR reactions and a 783 bp Salmonella gene target was amplified using OmniTaq in the presence of PVP alone or PVP coupled with PEC-1.

FIG. 9 is a series of line graphs and agarose gel images showing PCR detection of samples spiked with Salmonella and enriched. FIG. 9A shows real-time PCR without enhancer and 1×SYBR. FIG. 9B shows real-time PCR with 8% PVP enhancer and 5×SYBR. Further information regarding methodology is presented in Example 5.

FIG. 10 is a line graph showing real-time PCR detection of salmonella in cheese samples either without enhancer at 1×SYBR or with 8% PVP at 5×SYBR. Further information regarding methodology is presented in Example 5.

FIG. 11 is a series of agarose gel images showing PCR detection of Salmonella DNA in chocolate samples containing 5×SYBR, and PVP at a concentration of 0, 2%, 4%, 6%, 8%, 10%, 12%, 14%, 16%, or 18%. Further information regarding methodology is presented in Example 5.

FIG. 12 is a series of agarose gel images showing PCR detection of Salmonella DNA in samples containing 400 μg of chocolate (first enriched for competing microflora and then spiked with Salmonella DNA) with 8% PVP, PEC, or 8% PVP and PEC. Further information regarding methodology is presented in Example 6.

FIG. 13 is a series of agarose gel images showing PCR detection of Salmonella DNA in samples containing 200 μg of chocolate and 0.2% milk, 0.16% casein, 0.16% BSA, or 0.16% casiene and PEC (lane 1), with each subsequent lane doubling the protein concentration. All reactions contained template (salmonella cells), salmonella-specific primers, dntps, and OmniTaq-LA polymerase. Reactions were cycled for 95° for 8′, then 33 times [95° 50″, 66° 1′, 68° 6′]. Further information regarding methodology is presented in Example 7.

FIG. 14 is a series of agarose gel images showing PCR detection of Salmonella DNA in samples containing crushed potato peel in water. Each lane represents decreasing two-fold dilutions of crushed potato peel in water samples added to PCR reactions. Samples contained 0.4% casein (FIG. 14A) or 8% PVP (FIG. 14B). Further information regarding methodology is presented in Example 8.

FIG. 15 is a series of agarose gel images showing PCR detection of soybean cyst nematode DNA in soil samples containing humic acid and a polyphenolic inhibitor. Reactions contained a fixed amount of soil and 0%, 2.5%, 5%, 7.5%, 10%, or 12.5% PVP.

FIG. 16 shows an image of a gel of a 250 bp target of Lambda DNA amplified with 0.2 μl Omni Klentaq in the presence of 40 ng, 13.3 ng, 4.4 ng, 1.5 ng and 0.5 ng humic acid (lanes 1-5). Control reactions (lanes 6) contained no humic acid. The reactions were performed without additives, or in the presence of 9% PVP or 0.4% casein. Lanes M are the DNA ladder. The amplified products were resolved in a 2% ethidium bromide stained agarose gel.

FIG. 17 is an image of a gel showing direct amplification of HCV gene from dried blood spot on FTA card and paper card. A 224 bp HCV gene was directly amplified from a dried blood spot on FTA and general paper card. Legend: F, FTA card; P, paper card; R, RNA (positive control); N, negative control.

FIG. 18 is an image of a gel showing direct amplification of ribosomal 16s gene from plants. A 1.2 mm piece of soybean or tomato leaf was applied in a 25 μl PCR reaction and a 365 bp gene was directly amplified from these samples using OmniTaq and Omni Klentaq in the presence or absence of PEC-P. Legend: Lanes 1, 3, 5, 7, tomato; lanes 2, 4, 6, 8, soybean.

FIG. 19 shows a series of images of gels of a 320 bp target of the human b-actin gene amplified in a 50 μl PCR reaction with 0.5 μl Omni Klentaq enzyme in the presence of 1, 2, 4, 8, 12, and 16 μl of a crude extract from tobacco leaves (lanes 2-7). Control reactions (lanes 1) contained no plant tissue extract. The reaction in lane 1 with no enhancers was compromised due to the high volume of enzyme, the volume consistent with that needed for the inhibitor reactions. This set of reactions was preformed with no enhancer (top left panel), in the presence of PEC-1 (top right panel), or in the presence of PEC-1 supplemented with PVP to a final concentration of 4% in the reaction (bottom panel). Lanes M are the DNA ladders. The amplified products were analyzed in a 1.5% ethidium bromide stained agarose gel.

FIG. 20 shows profiles of direct STR genotyping of crude samples containing bile salts. FIG. 20A shows the profile using the DNAP technology protocol and FIG. 20B shows the profile using the PowerPlex 16HS Kit (Promega). OmniTaq and PEC-PVP enhancer cocktail were used for STR genotyping with the Promega Power Plex 16 kit primers, using 1 ng human DNA in the presence of 5 μl bile salts extract (12 μg/μl) and no DNA extraction. In control reactions the complete PP16HS kit was used (se e.g., FIG. 20B). Human Short Tandem Repeats (STR) in 16 loci were directly amplified from samples containing bile salts using OmniTaq in the presence of PEC-P (PEC-PVP) as an enhancer. The PowerPlex 16 HS kit from Promega was included as a comparison.

FIG. 21A and FIG. 21B show profiles of direct STR genotyping of crude human DNA samples in the presence of indigo dye using the DNAP Technology protocol and the Life Tech ID-PLUS kit, respectively. OmniTaq and PEC-PVP enhancer cocktail were used for Short Tandem Repeat (STR) genotyping with the ID-PLUS kit primers, using 1 ng human DNA in the presence of 50 μg indigo dye. In control reactions the complete ID-PLUS kit was used (see e.g., FIG. 21B).

FIG. 22 shows profiles of direct STR genotyping of crude human DNA samples in the presence of indigo dye using the DNAP Technology protocol and the Promega PP16HS kit, respectively. OmniTaq and PEC-PVP enhancer cocktail were used for STR genotyping with the Promega Power Plex 16 kit primers, using 1 ng human DNA in the presence of 200 μg indigo dye. In control reactions the complete PP16HS kit was used (see e.g., FIG. 22B).

FIG. 23 shows profiles of direct STR genotyping of crude human DNA samples in the presence of tannins using the DNAP Technology protocol and the Life Tech ID-PLUS kit, respectively. CesiumTaq and PEC-PVP enhancer cocktail were used for STR genotyping with the ID-PLUS kit primers, using 1 ng human DNA in the presence of 4 μg tannins. In control reactions the complete ID-PLUS kit was used (see e.g., FIG. 23B).

### DETAILED DESCRIPTION OF THE INVENTION

The present disclosure is based at least in part upon the discovery that casein or PVP can overcome inhibitory effects of chocolate in nucleic acid amplification reactions. The present disclosure is also based at least in part upon the discovery that casein or PVP can overcome inhibitory effects of samples (e.g., food, soil, or blood samples) containing inhibitory substances in nucleic acid amplification reactions. In various embodiments, enhancer compositions including casein or PVP can reduce or eliminate PCR inhibitory effects of PCR inhibitors, such as polyphenols, in samples such as chocolate, potato skins, tea, berries, beer, wine, olive oil, walnuts, or peanuts. In other embodiments, enhancer compositions including casein or PVP can reduce or eliminate PCR inhibitory effects of PCR inhibitors, such as polyphenols, in samples such as soil. In various embodiments, enhancer compositions including casein or PVP can reduce or eliminate inhibitory effects of elevated levels of dyes used in, for example, real-time PCR reactions. As shown herein, PCR detection of microbial pathogens, such as Salmonella, fail to work in polyphenol-containing PCR assays, due to the inhibition of the reaction by the polyphenols (or high fluorescence dye concentration).

The disclosure of PCT/US09/66868, published as WO 2010/065924, is incorporated herein by reference. Any and all compositions or methods described herein can be incorporated into or practiced with any of the compositions or methods disclosed in WO 2010/065924.

Provided herein are methods and compositions for improving performance in chocolate or other polyphenol-containing amplification reactions, (e.g., PCR, qPCR, RT-PCR, real-time RT-PCR) through addition of an enhancer composition including casein or PVP.

Enhancer compositions and methods described herein can allow for dilution of reagents included in several commercial rapid detection systems (e.g., primers, dNTPs, and dyes), leading to substantial cost savings. Enhancer compositions and methods described herein can allow for shortening or removing a cultural enrichment step or eliminating or substantially eliminating sample preparation, thereby reducing overall time-to-detect. Enhancer compositions and methods described herein can allow for elimination of dilution of the nucleic acid containing sample without loss of accuracy of the assay.

One aspect provides enhancer compositions including casein or PVP, useful in nucleic acid amplification reactions for overcoming inhibitory effects of PCR inhibitors, such as polyphenols, contained in the sample. Enhancer compositions including casein or PVP can be used in conjunction with other PCR enhancers, such as PEC or PEC-plus to further improve performance of nucleic acid amplification reactions.

Another aspect provides use of enhancer compositions including casein or PVP to overcome inhibitory effects of PCR inhibitors, such as polyphenols, in nucleic acid amplification reactions. Such an approach not only enhances conventional PCR but can also improve RT-PCR or real-time PCR performance with, for example, SYBR green fluorescence detection and TaqMan assay.

Thus is provided a valuable tool for an improved, low-cost, fast, and sensitive PCR detection of nucleic acids in samples such as chocolate or other PCR inhibitor-containing samples, such as samples containing polyphenols.

Enhancer Composition

An enhancer composition of the present invention can include one or more of casein or PVP, or a modification thereof. In some embodiments and enhancer composition of the present disclosure can further include at least one of trehalose, carnitine, a nonionic detergent, or heparin. Any of the compositions described in WO 2010/065924, incorporated herein by reference, can further include casein or PVP, or a modification thereof, according to concentrations described herein.

As demonstrated herein, an enhancer composition comprising casein or PVP, or a modification thereof, can reduce or eliminate inhibitory effects of PCR inhibitor-containing samples (e.g., polyphenol-containing samples) on polymerase amplification reactions, such as PCR, RT-PCR, real time PCR, or real time RT-PCR. For example, an enhancer composition comprising casein or PVP, or a modification thereof, can reduce or eliminate inhibitory effects of chocolate on polymerase amplification reactions.

Component concentrations herein are expressed in terms of concentration in the final amplification reaction mixture volume, unless indicated otherwise. In some embodiments, an enhancer composition is in concentrated form such that when added to a reaction mixture volume, the concentration of components recited herein results. For example, an enhancer composition can be 2× (two-fold concentrated), such that the component concentration in the concentrated enhancer composition is two-times higher than the actual final concentration of the enhancer components in a reaction mixture.

Casein

An enhancer composition of the present disclosure can include casein. As demonstrated herein, casein can be included in an amplification reaction mixture so as to reduce or eliminate inhibitory effects of a polyphenol. For example, casein can be included in an amplification reaction mixture so as to reduce or eliminate inhibitory effects of chocolate.

While being under no obligation to supply a mechanism, and in no way limiting the present invention, it is presently thought that casein can bind, precipitate, or encapsulate polyphenolic compounds in chocolate, or other samples containing PCR inhibitors, such as polyphenols.

A casein of an enhancer composition can be casein or a salt thereof, such as a casein sodium salt. A casein of an enhancer composition can be an αS1 casein, an αS2 casein, a β casein, a κ casein, or a paracasein.

A casein can be included in an amplification reaction mixture at a concentration of at least about 0.05%. Casein can be included in an amplification reaction mixture at a concentration of up to about 2.5%. Casein can be included in an amplification reaction mixture at a concentration of at least about 0.05% up to about 2.5%. For example, casein can be included in an amplification reaction mixture at a concentration of about 0.1%, about 0.5%, about 1%, about 1.5%, about 2%, or about 2.5%. As another example, casein can be included in an amplification reaction mixture at a concentration of at least about 0.1% up to about 1%.

As another example, casein can be included in an amplification reaction mixture at a concentration of at least about 0.1% up to about 0.8%. As another example, casein can be included in an amplification reaction mixture at a concentration of at least about 0.1%, at least about 0.2%, at least about 0.3%, at least about 0.4%, at least about 0.5%, at least about 0.6%, at least about 0.7%, or at least about 0.8%. As another example, casein can be included in an amplification reaction mixture at a concentration of about 0.1%, about 0.2%, about 0.3%, about 0.4%, about 0.5%, about 0.6%, about 0.7%, or about 0.8%. As reported herein, casein included in an amplification reaction mixture at a concentration of about 0.4% has been shown to produce optimal amplification in a chocolate-containing sample.

Casein concentrations discussed above are expressed in terms of concentration in a final amplification reaction mixture volume. Calculation of a corresponding concentration in an isolated (and concentrated) enhancer composition is within the skill of the art. The above concentrations of casein can be combined with any of the concentrations of PVP (or a modification thereof) or other enhancer component concentrations (e.g., trehalose, carnitine, a nonionic detergent, or heparin) discussed herein.

PVP in Amplification Reactions

As described herein, polyvinylpyrrolidone (PVP), or a modification thereof, can be included in an amplification reaction mixture so as to reduce or eliminate inhibitory effects of a polyphenol. For example, PVP can be included in an amplification reaction mixture so as to reduce or eliminate inhibitory effects of chocolate.

A modified PVP includes, but is not limited to polyvinylpolypyrrolidone (PVPP), which is an insoluble highly cross-linked modification of PVP. It will be understood that disclosure herein related to PVP can be adapted to PVPP.

PVP can be included in an amplification reaction mixture at a concentration of at least about 0.1%. PVP can be included in an amplification reaction mixture at a concentration of up to about 25%. PVP can be included in an amplification reaction mixture at a concentration of at least about 0.1% up to about 25%. For example, PVP can be included in an amplification reaction mixture at a concentration of about 1%, about 5%, about 10%, about 15%, about 20%, or about 25%. As another example, PVP can be included in an amplification reaction mixture at a concentration of at least about 1% up to about 15%. PVP can be included in an amplification reaction mixture at a concentration of at least about 1% up to about 10%. Each of the above concentrations apply also to a modified PVP, such as PVPP.

As another example, PVP can be included in an amplification reaction mixture at a concentration of at least about 8% up to about 10%. As another example, PVP can be included in an amplification reaction mixture at a concentration of at least about 8%, at least about 8.5%, at least about 9%, at least about 9.5%, or at least about 10%. As another example, PVP can be included in an amplification reaction mixture at a concentration of about 8%, about 8.5%, about 9%, about 9.5%, or about 10%. Each of the above concentrations apply also to a modified PVP, such as PVPP. As reported herein, PVP included in an amplification reaction mixture at a concentration of about 8% to about 10% has been shown to produce optimal amplification in a chocolate-containing sample.

PVPP can be included in an amplification reaction mixture at a concentration of at least about 0.1%. PVPP can be included in an amplification reaction mixture at a concentration of up to about 25%. PVPP can be included in an amplification reaction mixture at a concentration of at least about 1% up to about 25%. For example, PVPP can be included in an amplification reaction mixture at a concentration of about 1%, about 5%, about 10%, about 15%, about 20%, or about 25%. As another example, PVPP can be included in an amplification reaction mixture at a concentration of at least about 1% up to about 15%. As another example, PVPP can be included in an amplification reaction mixture at a concentration of at least about 1% up to about 10%. As another example, PVPP can be included in an amplification reaction mixture at a concentration of at least about 1% up to about 5%. As another example, PVPP can be included in an amplification reaction mixture at a concentration of about 1%, about 2%, about 3%, about 4%, or about 5%.

PVP (and modifications thereof) concentrations discussed above are expressed in terms of concentration in a final amplification reaction mixture volume. Calculation of a corresponding concentration in an isolated (and concentrated) enhancer composition is within the skill of the art. The above concentrations of PVP (or a modification thereof) can be combined with any of the casein concentrations or other enhancer component concentrations (e.g., trehalose, carnitine, a nonionic detergent, or heparin) discussed herein.

Culture Media

As described herein, polyvinylpyrrolidone (PVP), or a modification thereof (e.g., PVPP) or casein can be included in a microorganism culture media. For example, PVP or casein can be included in a bacterial growth media so as to reduce or eliminate inhibitory effects. As another example, PVP or casein can reduce inhibition of Salmonella growth in chocolate samples, allowing more colonies to grow by relieving the inhibition caused by chocolate.

PVP or a modified PVP can be as discussed above. Casein can be as discussed above.

Culture of microorganisms and media for use therein are well known; see e.g., Atlas 2010 Handbook of Microbiological Media, Fourth Edition, CRC Press, ISBN-10: 1439804060). Except as otherwise noted herein, therefore, the process of the present disclosure can be carried out in accordance with such processes.

PVP can be included in a solid growth media mixture at a concentration of at least about 0.1%. PVP can be included in a solid growth media mixture at a concentration of up to about 25%. PVP can be included in a solid growth media mixture at a concentration of at least about 0.1% up to about 25%. For example, PVP can be included in a solid growth media mixture at a concentration of about 1%, about 5%, about 10%, about 15%, about 20%, or about 25%. As another example, PVP can be included in a solid growth media mixture at a concentration of at least about 1% up to about 15%. PVP can be included a solid growth media mixture at a concentration of at least about 1% up to about 10%. Each of the above concentrations apply also to a modified PVP, such as PVPP.

As another example, PVP can be included in a liquid growth media mixture at a concentration of greater than 0% up to about 4%. As another example, PVP can be included in a growth media mixture at a concentration of at least about 0.5%, at least about 1%, at least about 1.5%, at least about 2%, at least about 2.5%, at least about 3%, at least about 3.5%, or about 4%. Each of the above concentrations apply also to a modified PVP, such as PVPP.

Furthermore, PVP can be used to inhibit microorganism growth where PVP or modification thereof is included in a liquid growth media (see e.g., Example 4) For example, a PVP concentration greater than about 4%, such as greater than about 5%, greater than about 6%, greater than about 7%, greater than about 80%, or more can inhibit microorganism growth where PVP or modification thereof is included in a liquid growth media n (see Example 4).

Casein can be included in a solid growth media mixture at a concentration of at least about 0.1%. Casein can be included in a solid growth media mixture at a concentration of up to about 10%. Casein can be included in a solid growth media mixture or a liquid growth media at a concentration of at least about 0.1% up to about 10%. For example, casein can be included in a growth media (e.g., solid or liquid growth media) mixture at a concentration of about 1%, about 2%, about 3%, about 4%, about 5%, about 6%, about 7%, about 8%, about 9%, or about 10%.

A microorganism can be any microorganism that experiences growth inhibition in a growth media. For example, the microorganism can be Salmonella.

PEC and Additional Components

As described herein, casein or PVP can be included in an amplification reaction mixture in conjunction with a PEC or PEC plus enhancer composition so as to reduce or eliminate inhibitory effects of a polyphenol. For example, casein or PVP and a PEC enhancer composition can be included in an amplification reaction mixture so as to reduce inhibitory effects of chocolate.

A PEC or PEC plus enhancer can be according to any enhancer composition described in PCT App No. U.S.09/66868, filed Dec. 4, 2009, published as WO 2010/065924, and corresponding U.S. App Ser No. 13/133,150, filed Oct. 10, 2011, each of which is incorporated herein by reference. Any and all compositions or methods described herein can be incorporated into or practiced with any of the compositions or methods disclosed in WO 2010/065924 or U.S. application Ser No. 13/133,150.

For example, an enhancer composition including casein or PVP (or a modification thereof) at a concentration described herein can further include about 0.1 to about 0.8 M trehalose, about 0.1 M to about 1.5 M L-carnitine, and about 0.01 to about 8% nonionic detergent, such as Brij-58 or NP-40. As another example, an enhancer composition including casein or PVP (or a modification thereof) at a concentration described herein can further include about 0.6 M D-(+)-trehalose, about 0.48 M L-carnitine, and about 0.8% NP-40 (PEC).

A PEC enhancer can be included in an amplification reaction mixture where a sample is an enriched sample. Used in conjunction with casein or PVP, a PEC enhancer can omit one or more components. For example, when used in conjunction with casein or PVP, a PEC enhancer can omit a detergent component. When used in conjunction with casein or PVP, a PEC enhancer can comprise a detergent at a concentration of about 0 to about 1%. For example, when used in conjunction with casein or PVP, a PEC enhancer can comprise a detergent, such as NP40 or Brij, at a concentration of about 0.25% to about 0.5%. A detergent can be supplied as a component of an enhancer, such as a PEC enhancer, or as a component of a reaction buffer.

An enhancer composition described herein can include one or more additional components selected from betaine, DTT, dimethyl sulfoxide (DMSO), BSA, glycerol, formamide, ammonium ions (e.g., (NH)₄SO₄), polyethylene glycol, and tetramethyl ammonium chloride.

An enhancer composition described herein can be employed along with other convention amplification reaction enhancers (in the same or different compositions) to further improve amplification (e.g., BAX, DuPont; iQ-check, Biorad; MasterAmp™ 10×PCR Enhancer, Epicentre Biotechnologies; TaqMaster PCR Enhancer, MasterTaq Kit, PCR Extender System, 5 PRIME GmbH; Hi-Spec Additive, Bioline; PCRboost™, Biomatrica®; PCRX Enhancer System, Invitrogen; Taq Extender™ PCR Additive, Perfect Match® PCR Enhancer, Stratagene; Polymer-Aide PCR Enhancer, Sigma-Aldrich). For example, an enhancer composition can be combined with a PCR reaction mixture along with betaine (e.g., MasterAmp™ 10×PCR, Epicentre Biotechnologies). Generally, betaine alone is insufficient to overcome the inhibition of, for example, blood, polyphenols, or dye when used with conventional DNA polymerases. As another example, usage of another conventional amplification reaction enhancer can be according to manufacturer instructions.

Addition of Enhancer

An enhancer composition described herein can be introduced into an amplification reaction before, during, or after introduction of a target nucleic acid or a sample including or thought to include a target nucleic acid. An enhancer composition can be introduced into an amplification reaction before, during, or after introduction of primers. An enhancer composition can be introduced into an amplification reaction before, during, or after introduction of a polymerase enzyme. An enhancer composition can be introduced into an amplification reaction before, during, or after introduction of deoxynucleoside triphosphates. An enhancer composition can be introduced into an amplification reaction before, during, or after introduction of a buffer solution. An enhancer composition can be introduced into an amplification reaction before, during, or after introduction of divalent cations, such as magnesium or manganese ions. An enhancer composition can be introduced into an amplification reaction before, during, or after introduction of monovalent cations, such as potassium ions.

Target Nucleic Acid

A target nucleic acid of a PCR inhibitor-containing sample (e.g., a polyphenol-containing sample) can be any target nucleic acid of interest. For example, a target nucleic acid can be associated with or a part of a pathogen, such as a bacterial pathogen or a viral pathogen. As another example, a target nucleic acid can be associated with or a part of a bacterial pathogen, such as Salmonella, Listeria, or Toxoplasma. As another example, a target nucleic acid can be associated with or a part of a Salmonella.

Amplification Reactions

An enhancer composition described herein can be used in conjunction with a variety of nucleic acid amplification processes well known in the art (see e.g., Dorak (2006) Real-Time PCR, Taylor & Francis, ISBN 041537734X; Bustin, ed. (2004) A-Z of Quantitative PCR, International University Line, ISBN 0963681788). Examples of nucleic acid amplification processes for use with the enhancer composition of the present invention include, but are not limited to, Allele-specific PCR; Assembly PCR or Polymerase Cycling Assembly; Asymmetric PCR; Linear-After-The-Exponential-PCR; Helicase-dependent amplification; Hot-start PCR; Intersequence-specific PCR; Inverse PCR; Ligation-mediated PCR; Methylation-specific PCR; Miniprimer PCR; Multiplex Ligation-dependent Probe Amplification; Multiplex-PCR; Nested PCR; Overlap-extension PCR; Quantitative PCR; Quantitative End-Point PCR; Quantitative Real-Time PCR; RT-PCR (Reverse Transcription PCR); Solid Phase PCR; Thermal asymmetric interlaced PCR; Touchdown PCR; PAN-AC; Universal Fast Walking; Long PCR; Rapid Amplified Polymorphic DNA Analysis; Rapid Amplification of cDNA Ends (RACE); Differential Display PCR; In situ PCR; High-Fidelity PCR; PCR and DNA Sequencing (cycle sequencing).

For example, PCR is commonly carried out in a reaction volume of about 10-200 μl in small reaction tubes (about 0.2-0.5 ml volumes) in a thermal cycler. An enhancer composition can be combined with the PCR reaction mixture at any step including, initialization, denaturation, annealing, extension/elongation, final elongation, or hold. Preferably, the enhancer composition is combined with a PCR reaction mixture so as to be present during extension and elongation.

The use of an enhancer composition described herein generally does not require any, or substantial, changes in a typical protocol, other than, for example, combining the enhancer composition and the reaction mixture. Thus, compositions and methods described herein can be applied to improve the nucleic acid detection in any standard PCR protocol with, for example, samples containing PCR inhibitors, such as polyphenols.

Provided herein is a method for improving performance in PCR amplification of a heterogeneous mixture of nucleic acid templates. The heterogeneous mixture can include endogenous DNA or RNA or exogenous DNA or RNA (e.g., from a pathogen). Such a method generally includes forming an assay mixture from nucleic acid templates of varying G+C content; an enhancer composition described herein; and at least one polymerase. The assay mixture is then reacted so as to amplify the nucleic acid templates.

Also provided is a method for improving performance in real-time reverse transcriptase (RT) PCR amplification of an RNA target. An enhancer composition described herein can be used in a variety of RT-PCR protocols known to the art (see e.g., King and O'Connel (2002) RT-PCR Protocols, 1^{st} Ed., Human Press, ISBN-10 0896038750). It is noted that reverse transcriptase (RT) PCR is not to be confused with real-time polymerase chain reaction (Q-PCR), which is sometimes (incorrectly) abbreviated as RT-PCR in the art. In RT-PCR, an RNA strand is first reverse transcribed into its DNA complement (complementary DNA, or cDNA) using the enzyme reverse transcriptase, and the resulting cDNA is amplified using traditional PCR. Applications of RT-PCR include, but are not limited to, detection of RNA virus pathogens; analysis of mRNA expression patterns of certain genes related to various diseases; semiquantitative determination of abundance of specific different RNA molecules within a cell or tissue as a measure of gene expression; and cloning of eukaryotic genes in prokaryotes. A target RNA in an RT-PCR reaction including an enhancer composition can be, for example, an RNA of viral origin or an RNA of cellular origin. An RT-PCR reaction can comprise at least one polymerase, or preferably a mixture of polymerases as discussed above, and at least one reverse transcriptase. Selection of appropriate polymerases and reverse transcriptases is within the skill of the art. An enhancer composition can be combined with the RT-PCR reaction mixture at any step.

Also provided is a method for improving performance in dye-containing amplification reactions through addition of an enhancer composition described herein. Enhancer compositions and dyes are discussed further below.

Also provided is a method for improving performance in polyphenol-containing amplification reactions (e.g., PCR, qPCR, RT-PCR, real-time RT-PCR) through addition of an enhancer composition described herein. An enhancer composition can improve performance in the presence of PCR inhibitors, such as polyphenols. PCR inhibitors, such as polyphenols, and samples comprising such are further discussed below.

Polyphenol resistance can be readily determined by assays described herein and know in the art. As shown herein, a full-length Taq enzyme is completely inhibited at a chocolate concentration of more than ˜0.04 μg/μl chocolate in a sample, whereas a resistant polymerase, such as KT10 or FLAC2 can tolerate ˜0.16 μg/μl chocolate. Based on guidance of the present disclosure, the level of polyphenol resistant of a polymerase, and the known or estimated polyphenol concentration of a sample, one of ordinary skill can determine and amount or concentration of an enhancer composition described herein needed in an amplification reaction to reduce or eliminate polyphenol inhibitory effects. An enhancer composition can be used in conjunction with mutant polymerase enzymes and processes described in U.S. Pat. No. 7,462,475; US App Pub No. 2009/0170060; and WO/2008/034110, each incorporated herein by reference.

Also provided is a method for improving performance in amplification reactions (e.g., PCR, qPCR, RT-PCR, real-time RT-PCR) containing inhibitors found in soil and soil extracts through addition of an enhancer composition described herein. Many polymerases are inhibited by substances found in soil, such as humic acid, which contains elevated polyphenol levels. Conventional DNA polymerase enzymes are inhibited at about 1 ng of humic acid per 50 μL reaction volume. Assays to determine the level of inhibitory substances in a sample and soil-resistance of a polymerase are known in the art. An enhancer composition can be used in conjunction with mutant polymerase enzymes and processes described in WO/2008/034110, incorporated herein by reference.

An enhancer composition described herein can be used in conjunction with compositions and processes described in U.S. Pat. No. 6,403,341, incorporated herein by reference. An enhancer composition can be used in conjunction with compositions and processes described in U.S. Pat. No. 7,393,635, incorporated herein by reference. An enhancer composition can be used in conjunction with compositions and processes described in US App Pub No. 2008/0262212, incorporated herein by reference.

Enhancer compositions or methods described herein can be used with a variety of commercial amplification systems. For example, an enhancer composition described herein can be used with a high-throughput detection system such DuPont Qualicon's BAX and Bio-Rad's iQ-Check systems.

An enhancer composition described herein can be used at any step or stage of a high-throughput detection system, such DuPont Qualicon's BAX and Bio-Rad's iQ-Check systems. For example, an enhancer composition system described herein can be used during enrich or regrow of samples. As another example, an enhancer composition system described herein can be included in or used with a lysis buffer. As another example, an enhancer composition system described herein can be combined with an enriched or regrown sample. As another example, an enhancer composition system described herein can be used in a heat lysis. As another example, an enhancer composition system described herein can be used in a first stage heat lysis. As another example, an enhancer composition system described herein can be used in a second stage heat lysis. As another example, an enhancer composition system described herein can be used in a cooling stage after heat lysis. As another example, an enhancer composition system described herein can be used with a lysate sample. As another example, an enhancer composition system described herein can be used in one or more cycling reactions of a lysate sample. As another example, an enhancer composition described herein can reduce or eliminate the need for lysis of a sample.

Polymerases

An enhancer composition described herein can be used in conjunction with a variety of polymerase enzymes or an amplification reaction a variety of polymerase enzymes. Suitable polymerases include, but are not limited to, those from polymerase families A, B, C, D, X, Y, and RT. Examples of polymerase enzymes for use with the enhancer composition of the present invention include, but are not limited to, T7 DNA polymerase; DNA Polymerase γ; E. coli DNA pol I; Thermus aquaticus pol I; Bacillus stearothermophilus pol I; DNA polymerase α; DNA polymerase β; DNA polymerase γ; DNA polymerase δ; DNA polymerase ε; DNA polymerase ζ; T4 polymerase; Phi29 polymerase; RB69 polymerase; DNA Polymerase III; polymerase pol β; polymerase pol σ; polymerase pol λ; polymerase pol μ; terminal deoxynucleotidyl transferase (TdT); polymerase Po14; translesion synthesis (TLS) polymerases; Pol I; Pol II; Pol III; Pol IV; Pol V; reverse transcriptase polymerase (e.g., AMV Reverse Transcriptase, M-MLV Reverse Transcriptase, M-MLV Reverse Transcriptase, and RNase H Minus); mutations or truncations thereof, and combinations thereof.

An enhancer composition described herein can be used in conjunction with one or more thermostable polymerase including, but not limited to, Taq, Tfl, Tth, Tli, Pfu, mutations or truncations thereof, and combinations thereof.

An enhancer composition described herein can be used in conjunction with one or more polymerases selected from OmniTaq (DNA Polymerase, Inc., St. Louis, Mo.), Omni Klentaq (DNA Polymerase, Inc., St. Louis, Mo.; see KT-10 in U.S. Pat. No. 7,462,475); Omni Klentaq-LA (a 50:1 mixture of Omni Klentaq with Deepven); a truncated version of wild type Taq DNA polymerase with point mutations lacking 5′→3′ exonuclease activity (see U.S. Pat. No. 7,462,475, incorporated herein by reference); wild type Taq; FastStart Taq; JumpStart Taq; HotStart Plus Taq; AmpliTaq Gold; and combinations thereof.

An enhancer composition described herein can be used in conjunction with a mutant polymerase enzymes and processes described in U.S. Pat. No. 7,462,475 and US App Pub No. 2009/0170060, each incorporated herein by reference. An enhancer composition described herein can be used in conjunction with mutant polymerase enzymes and processes described in WO/2008/034110, incorporated herein by reference.

A polymerase of an amplification reaction can be, for example a Taq polymerase or mutant thereof. Exemplary mutant polymerases for use in methods described herein include, but are not limited to Klentaq (KT) 1, FLAC2, FLAC3, FLAC4,

The enzymes specified are mutants of full-length Taq or Klentaq1 that were specifically selected for inhibition-resistance. Klentaq1 is an N-terminal deletion of Taq which confers higher fidelity and thermostability to the enzyme (Barnes W M, 1992.) Enzymes such as Klentaq (KT) 1, FLAC2, FLAC3, and FLAC4 have two point mutations (E626K and I707L) that confer cold-sensitivity, which can provide for automatic hot-start for PCR. In addition, these mutants have an additional amino acid substitution that can provide inhibition resistance. FLAC2, FLAC3, and FLAC4 are the mutants of full-length Taq, with the mutations, E708Q, E708N, and E7081, respectively. The mutant of Klentaq1, known as Klentaq10, has an E708K substitution. It is presently thought the mutant enzymes hold more tightly to the target DNA, effectively prying inhibitors off the template as they go.

A polymerase of an amplification reaction can include a mixture of polymerases. For example, accurate (LA) counterparts are enzyme mixtures containing a small amount of Deep Vent DNA polymerase (New England Biolabs), which has 3′ to 5′ exonuclease activity. Such mixtures can increase fidelity and allows for amplification of longer targets (see e.g., Barnes W M, 1994, PNAS). As another example, while KT1 is not compatible with TaqMan chemistry (Applied Biosystems) due to its 5′ deletion, an enzyme mixture of KT1 and another polymerase can be used in TaqMan.

Samples

An enhancer composition described herein can reduce or eliminate PCR inhibitory effects of chocolate or PCR inhibitors, such as polyphenols, in a sample. A sample can be an enriched sample or a non-enriched sample.

Concentration of a target nucleic acid in a sample can be according to methods known in the art, such as filtration, centrifugation, extraction and precipitation, column-binding s (see e.g., Liu 2008), aqueous two-phase system of PEG and Dextran 40 (Lantz P, et al.) and the use of alcohol precipitation of DNA in the presence of NaI (Makino S, et al.). It is understood that an enhancer composition can be introduced to a sample at any stage of any of the above described processes. For example, a chocolate-containing sample can be centrifuged and an enhancer described herein can be added to the sample prior to centrifugation or to any resulting fraction of the sample.

Samples containing a target nucleic acid and PCR inhibitors, such as polyphenols, for use low-throughput situations can be processed according to methods known in the art, such as lysis, extraction, and precipitation; chemical extraction with phenol/chloroform; or spin columns composed of glass beads with optional iron or pathogen-specific primers or antibodies in or on the beads. It is understood that an enhancer composition can be introduced to a sample at any stage of any of the above described processes. It is understood that reagents known to be inhibitory to PCR are generally avoided during processing steps.

Samples containing a target nucleic acid and PCR inhibitors, such as polyphenols, for use high-throughput situations can be processed according to relatively simple processes. For example, DuPont Qualicon's BAX and Bio-Rad's iQ-Check systems can use simple dilution of a cultured sample into a protease/lysis solution, followed by an incubation. An aliquot of this solution can then be added directly to a PCR reaction. But such approach can be insufficient for samples containing inhibitory substances such as polyphenols.

Compositions and methods described herein can allow for elimination and substantial elimination of an enrichment step for sample preparation. Eliminating an enrichment step can significantly reduce the time to detection, as it eliminates a conventional overnight cultural enrichment step. Eliminating an enrichment step can provide the ability to generate data about initial pathogen concentration, which can help determine the severity of an outbreak (e.g., quantitative PCR). An enhancer composition can include one or more of carnitine, trehalose, and a non-ionic detergent (e.g., a PEC enhancer plus casein or PVP, or a modification thereof).

High-throughput detection (e.g., DuPont Qualicon's BAX; Bio-Rad iQ-Check) can require additional sample preparation for non-enriched samples containing inhibitory substances such as polyphenols. Sample preparation can be according to, for example, PrepSEQ Rapid Spin sample preparation kit (Applied Biosystems, as part of the MicroSEQ Food Pathogen Detection Solution), which adds a spin-column step to sample preparation described above. Sample preparation can be according to, for example, PrepMan Ultra sample preparation reagent, which can require a centrifugation step without spin columns. Sample preparation can be according to, for example, Assurance GDS system (BioControl), which uses a higher-tech sample preparation with antibody-labeled beads. Such additional processing in conjunction with compositions and methods described herein can provide further improved rapid detection from inhibitory samples.

Compositions and methods described herein can reduce or eliminate inhibitory effects of PCR inhibitors, such as polyphenols, in a nucleic acid amplification reaction. For example, including casein or PVP in an amplification reaction mixture can reduce or eliminate inhibitory effects of PCR inhibitors, such as polyphenols, occurring in samples such as chocolate, potato skins, tea, berries, beer, wine, olive oil, walnuts, or peanuts. As another example, including PVP in an amplification reaction mixture can reduce or eliminate inhibitory effects of polyphenolic-like humic acid occurring in soil samples. As another example, including PVP in an amplification reaction mixture can reduce or eliminate inhibitory effects of polyphenolic compounds occurring in plant samples.

Compositions and methods described herein can reduce or eliminate inhibitory effects of chocolate, or components thereof, in a nucleic acid amplification reaction. Many DNA polymerases are inhibited in convention amplification reactions by polyphenolic compounds and other substances found in chocolate. Conventional DNA polymerase enzymes are inhibited at concentrations as low as about 2 μg of crude chocolate per 50 μL reaction volume.

An enhancer composition described herein can improve performance in the presence of chocolate, cultured chocolate, or chocolate components. Cultured chocolate includes, but is not limited to, chocolate, cultural media components, and various bacteria at various concentrations. Chocolate includes, but is not limited to, the crude bean from the tree, Theobroma cacao, as well as substances derived from this bean, such as cocoa powder, and chocolate confections. Chocolate may also include chocolate extracts, and fractionated, or partially purified chocolate-containing samples. Cultural media components include, but are not limited to, non-fat dry milk, brilliant green dye, and, less preferably, casein, salts, sugars, and buffering agents.) Chocolate components include, but are not limited to, polyphenolic compounds and other secondary plant metabolites, proteins, nucleic acids, sugars, amino acids, fatty acids, mineral ions, and hormones. An enhancer composition described herein can be used in a PCR to aid amplification of a nucleic acid target in the presence of one or more such chocolate components.

An assay mixture can include a volume of chocolate, a mixture of chocolate and culture media, or an extract of chocolate. Enhancer compositions described herein, such as PVP, can be used in PCR to amplify a nucleic acid target in the presence of one or more such chocolate or chocolate culture media, or chocolate extract. An assay mixture can include a concentration of chocolate at least about 0.05 μg/μl up to about 20 μg/μl. For example, an assay mixture can include a concentration of chocolate at least about 0.05 μg/μl; at least about 0.1 μg/μl; at least about 1 μg/μl; at least about 2.5 μg/μl; at least about 5 μg/μl; at least about 7.5 μg/μl; at least about 10 μg/μl; at least about 12.5 μg/μl; at least about 15 μg/μl; at least about 17.5 μg/μl; or at least about 20 μg/μl.

Other PCR inhibitor-containing samples (e.g., polyphenol-containing samples) include potato skins, tea, berries, beer, wine, olive oil, walnuts, peanuts, and soil.

An assay mixture can include a volume of soil or soil extract. Enhancer compositions described herein, such as PVP, can be used in PCR to amplify a nucleic acid target in the presence of soil or soil extract. For example, an assay mixture can contain soil or soil extract at a concentration of at least about 1% up to about 90%; at least about 1% up to about 80%; at least about 1% up to about 70%; at least about 1% up to about 60%; at least about 1% up to about 50%; at least about 1% up to about 40%; at least about 1% up to about 30%; at least about 1% up to about 20%; or at least about 1% up to about 10% of a total volume of the assay mixture. As another example, an assay mixture can contain soil or soil extract at an equivalent amount that provides up to about 25 ng of humic acid per 50 μL reaction volume; up to about 20 ng of humic acid per 50 μL reaction volume; or up to about 10 ng of humic acid per 50 μL reaction volume.

An assay mixture can include a volume of blood or blood fraction. Whole blood generally comprises plasma, serum, and blood cells. Blood components include, but are not limited to, red blood cells, white blood cells (e.g., leukocytes or platelets, i.e., thrombocytes), plasma, serum, hemoglobin, water, proteins, glucose, amino acids, fatty acids, mineral ions, hormones, carbon dioxide, urea, and lactic acid. Enhancer compositions described herein, such as PVP, can be used in PCR to amplify a nucleic acid target in the presence of one or more such blood components. An enhancer composition described herein, such as a composition including PVP, can generally facilitate amplification in PCR assays (e.g., real-time PCR or real-time RT PCR) containing from about 1% to about 25% blood or blood component, such as plasma or serum, in the reaction mixture (vol/vol). For example, blood, blood plasma, or blood serum can comprise at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% of a total volume of a PCR assay mixture comprising an enhancer composition described herein. For example, whole blood or a blood component, such as plasma or serum, can comprise at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, or 25% of a total volume of a PCR assay mixture comprising an enhancer composition described herein.

An assay mixture can include a volume of plant or plant extract. Enhancer compositions described herein, such as PVP, can be used in PCR to amplify a nucleic acid target in the presence of one or more such plant or plant extracts. A sample containing plant or plant extract can contain condensed tannins, which can comprise up to about 50% of dry weight. Such a sample or fraction thereof can be included in an assay mixture. For example, an assay mixture can contain plant or plant extract at a concentration of at least about 1% up to about 90%; at least about 1% up to about 80%; at least about 1% up to about 70%; at least about 1% up to about 60%; at least about 1% up to about 50%; at least about 1% up to about 40%; at least about 1% up to about 30%; at least about 1% up to about 20%; or at least about 1% up to about 10% of a total volume of the assay mixture.

As shown herein, casein helps both Omni Klentaq and plain Taq in overcoming plant tissue PCR inhibitors. For example, a human DNA test target was amplified in the presence of increasing amounts of tobacco leaf crude extract, up to 16% extract in the final reaction volume. The tolerance of both Omni Klentaq and a plain Taq (NEB) to the plant inhibitors was increased at least 4-8 times in the presence of 0.6% Casein. Higher amounts of casein may allow even higher resistance to inhibition.

Exemplary plant material includes, but is not limited to, soybean, tomato, tobacco, or tea. For example, tea polyphenols, (real-time PCR) Taq can tolerate about 8 ng per 25 μl reaction volume, while OmniTaq could tolerate 17 ng per 25 μl reaction volume and OmniKlentaq could tolerate 34 ng per 25 μl reaction volume. An enhancer composition described herein can provide for amplification of a target nucleic acid in a sample containing up to about 300 ng of tea polyphenols per 25 μl reaction volume. For example, enhancer composition described herein can provide for amplification of a target nucleic acid in a sample containing up to about 50 ng, up to about 75 ng, up to about 100 ng, up to about 125 ng, up to about 150 ng, up to about 175 ng, up to about 200 ng, up to about 225 ng, up to about 250 ng, up to about 275 ng, or up to about 300 ng of tea polyphenols per 25 μl reaction volume. As another example, an assay mixture can contain plant or plant extract at an equivalent amount that provides up to about 25 ng of tannins per 50 μL reaction volume; up to about 20 ng of tannins per 50 μL reaction volume; or up to about 10 ng of tannins per 50 μL reaction volume. Concentrations of polyphenols discussed above can be extrapolated to other polyphenol-containing samples.

Assays to determine the level of inhibitory substances in a sample and resistance of a polymerase are known in the art. For example, polyphenolic content can be assessed according to volumetric titration (e.g., oxidizing agent such as permanganate), colorimetric assay (e.g., Porter's Assay, Folin-Ciocalteu reaction), antioxidant capacity of a fraction (e.g., TEAC assay, DPPH assay, ORAC assay, FRAP assay, lipoprotein oxidation inhibition assay), biosensor, or diode array detector-coupled HPLC.

An enhancer composition can be used in conjunction with mutant polymerase enzymes and processes described in WO/2008/034110, incorporated herein by reference.

Dye

As described herein, PVP can be included in an amplification reaction mixture, such as a realtime PCR reaction mixture, so as to reduce or eliminate inhibitory effects of a dye, such as SYBR green, Eva Green or LC-Green. As described herein, elevated levels of dye can be used in amplification reactions so as to improve detection in samples such as chocolate or other PCR inhibitor-containing samples (e.g., polyphenol-containing samples). But increased dye can be inhibitory to the polymerase reaction. An enhancer described herein can reduce or eliminate inhibitory effects of dye. An enhancer described herein can also reduce or eliminate inhibitory effects of PCR inhibitors, such as polyphenols, of the sample. Thus is provided is a method for improving performance in dye-containing amplification reactions through addition of an enhancer composition described herein.

An enhancer composition can be added to a real-time PCR (qPCR) reaction mixture to overcome inhibitory effects of dyes (e.g., fluorescent dyes) used in qPCR. For example, an enhancer composition can be added to a real-time PCR (qPCR) reaction mixture including a PCR-inhibitor-containing sample (e.g., a polyphenol containing sample) in which elevated levels of elevated levels of dyes are used to increase signal, where the enhancer reduces or eliminates inhibitory effects of both PCR inhibitors, such as polyphenols, and elevated dyes.

Dyes for use in the methods described herein include, but are not limited to, SYBR Green (Molecular Probes, Eugene, Oreg.), LC Green (Idaho Technology, Salt Lake City, Utah), PicoGreen (Molecular Probes, Eugene, Oreg.,), TOTO (Molecular Probes, Eugene, Oreg.), YOYO (Molecular Probes, Eugene, Oreg.) and SYTO9 (Molecular Probes, Eugene, Oreg.). Dye-resistance can be readily determined by assays known in the art.

An enhancer composition described herein can increase polymerase tolerance against increased concentrations of dyes. Such increased concentrations include, but are not limited to, up to about 0.5×, 1×, 1.5×, 2×, 2.5×, 3×, 3.5×, 4×, 4.5×, 5×, 5.5×, 6×, 6.5×, 7×, 7.5×, 8×, 8.5×, 9×, 9.5×, 10×, 15×, 20×, 25×, 30×, 35×, 40×, 45×, 50×, or even higher over the dye concentration conventionally used in the assay. As an example, X can be the standard manufacturers unit for dye concentration provided in a commercial product (e.g., SYBR Green, Molecular Probes, Eugene, Oreg.). For example, where the dye is SYBR Green, 1× can correspond to a concentration of about 1 μg/ml.

An enhancer composition including PVP can allow use of a dye, such as SYBR, in an amplification reaction mixture at a concentration of about 1× to about 20×. For example, PVP can allow use of a dye, such as SYBR, in an amplification reaction mixture at a concentration of about 5×, about 10×, about 15×, or about 20×. As another example, PVP can allow use of a dye, such as SYBR, in an amplification reaction mixture at a concentration of about 5×. PVP concentrations in an amplification reaction mixture comprising a dye, such as SYBR, can be according to those described above. Similarly, a modified PVP, such as PVPP, can be included in an amplification reaction mixture so as to reduce or eliminate inhibitory effects of a dye according to the above described conditions.

Kit

Another aspect of the invention is directed toward kits for performance enhancement of amplification reactions. Such kits can include an enhancer composition of the present invention and, in certain embodiments, instructions for administration. Such kits can also contain components for an amplification reaction, as described above. Preferably, a kit comprises an enhancer composition described herein, one or more polymerases (and optionally a reverse transcriptase), a buffer, and nucleotides.

Various embodiments of the kit can facilitate performance of the methods described herein, for example, PCR reactions. When supplied as a kit, the different components of the composition can be packaged in separate containers and admixed immediately before use. Components include, but are not limited to enhancer compositions described herein, primers, polymerases, deoxynucleoside triphosphates, buffer solution, divalent cations, monovalent cations, or combinations thereof. Such packaging of the components separately can, if desired, be presented in a pack or dispenser device which may contain one or more unit dosage forms containing the composition(s). The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain lyophilized agent(s) and in a separate ampule, sterile water or sterile saline, each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

General

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### PCR Amplification Reaction

This example describes PCR detection of Salmonella. 50 μl reactions were run on a Robocycler-40 with hot stop (Strategene). Reactions contained 1× buffer (50 mM Tris-Ph 9.2, 16 mM AmSO4, 3.5 mM MgCl2, and 0.1% Tween-20 (for all reactions except FLAC2 (OMNI TAQ), which had only an altered pH of 8.2 and 2.5 mM MgCl2), 200 μM each dNTP, 0.5 ng Salmonella DNA (LT2), 200 nM each of Salmonella primer (inv5-5 and inv5-3), and 0.4 μl Taq (NEB) or 0.1 μl Klentaq 1 (KT1), Klentaq (KT10), and FLAC2 (DNAP Polymerase, Inc.). In-house primers (Inv5-5: CTG GAA AAT GAA ATA CCG GAG GTT GA and Inv5-3: CGT AAT GTG GCG CTT GAG CAT GTA AC) were designed to amplify a 534 bp fragment of the inv gene within the SPI-1 pathogenicity island. This sequence is conserved across all subspecific groups (I to VII) of S. enterica and governs the ability of salmonellae to infect mammalian cells (Ochman H.).

### Example 2

### Chocolate Inhibition

This example demonstrates inhibitory effect of chocolate on PCR reactions. The example also demonstrates a base-line performance of various polymerases in the presence of chocolate in samples.

Increasing amounts of chocolate were added to PCR reactions containing Salmonella DNA. 50 μl reactions were run on a Robocycler-40 with hot stop (Strategene). Reactions contained 1× buffer (50 mM Tris-Ph 9.2, 16 mM AmSO4, 3.5 mM MgCl2, and 0.1% Tween-20 (for all reactions except FLAC2 (OMNI TAQ), which had only an altered pH of 8.2 and 2.5 mM MgCl2), 200 μM each dNTP, 0.5 ng Salmonella DNA (LT2), 200 nM each of Salmonella primer (inv5-5 and inv5-3), and 0.4 μl Taq (NEB) or 0.1 μl Klentaq 1 (KT1), Klentaq 10 (KT10, Omni Klentaq, DNAP Polymerase, Inc.), and FLAC2 (Omni Taq, DNAP Polymerase, Inc.). In-house primers (Inv5-5: CTG GAA AAT GAA ATA CCG GAG GTT GA and Inv5-3: CGT AAT GTG GCG CTT GAG CAT GTA AC) were designed to amplify a 534 bp fragment of the inv gene within the SPI-1 pathogenicity island. This sequence is conserved across all subspecific groups (I to VII) of S. enterica and governs the ability of salmonellae to infect mammalian cells (Ochman H.)

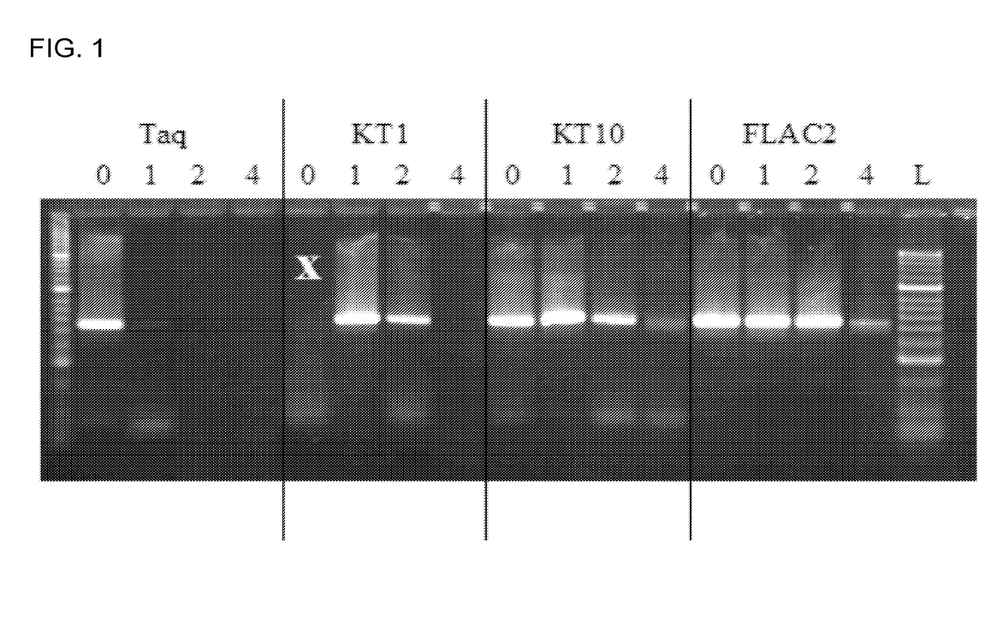

Chocolate was added in increasing concentrations at 1:256 (1 in FIG. 1), 1:128 (2 in FIG. 1), 1:64 (3 in FIG. 1) dilution of a ˜1 g/ml solution of chocolate chips in water. The “0” reaction contained no chocolate. Cycling conditions were 68° for 5″, 95° for 2′, then 35 cycles of [95° 40″, 66° 40″, 68° 2′].

Results showed reactions containing Taq were strongly inhibited by chocolate at all concentrations, with feint amplification at the 1:256 dilution (corresponding to ˜0.04 μg/μl chocolate). KT1 was able to tolerate some chocolate. The lane marked X in FIG. 1 is believed to be a spurious blank reaction. Inhibition resistant mutants KT10 and FLAC2 were able to tolerate relatively more chocolate, with FLAC2 showing amplication of the 1:64 dilution (corresponding to ˜0.16 μg/μl chocolate).

Real-time PCR reactions were run on an Opticon 2 (Bio-Rad). 25 μl reactions contained 0.5×SYBR Green, 1× buffer (50 mM Tris-Ph 8.2, 16 mM AmSO4, 2.5 mM MgCl2, and 0.1% Tween-20), 200 μM each dNTP, 2.5 ng Salmonella DNA (LT2), 200 nM each of Salmonella primer (inv5-5 and inv5-3), and 0.25 μl Taq (NEB) or 0.05 μl FLAC2 (DNAP Polymerase, Inc.) with 1.3 M betaine. Chocolate was added in increasing concentrations at 1, 1:512, 1:348, and so on up to 1:16 dilution of a ˜1 g/ml solution of chocolate chips in water. Cycling conditions were 95° for 1′, then 34 cycles of [95° 30″, 66° 30″, 68° 1′].

Results showed that FLAC2 with betaine tolerated nearly four times more chocolate than Taq.

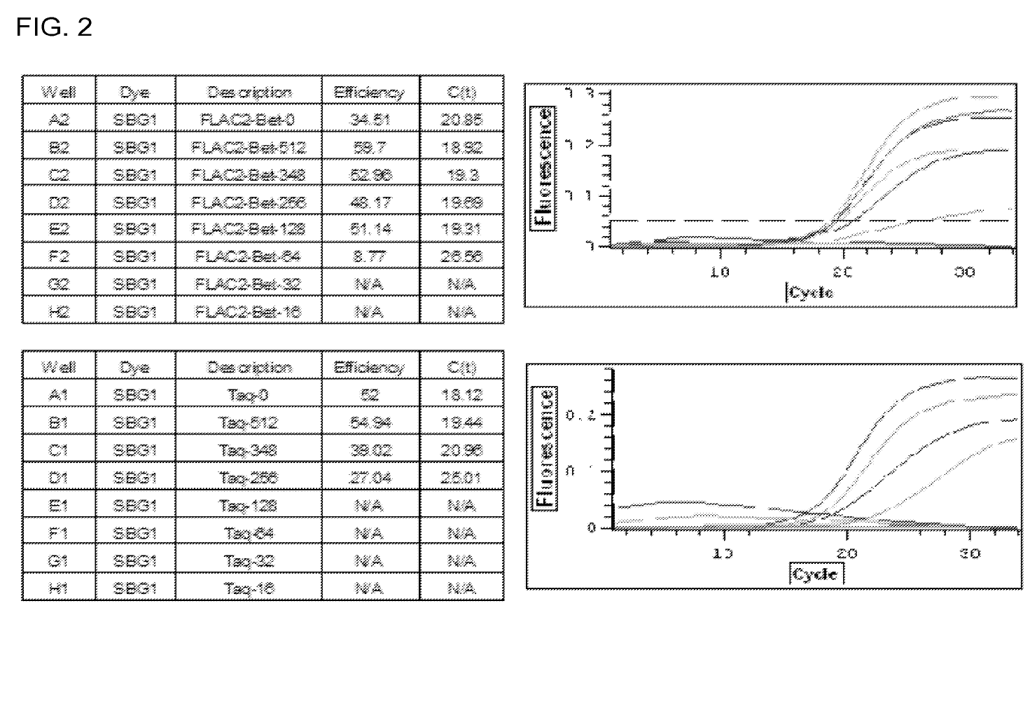

The above described results show that inhibition-resistant mutant enzymes are able to tolerate chocolate at an approximate concentration of 0.16 ug/ul, which was four times more than Taq. This approximates actual concentrations of chocolate in typical PCR reactions (e.g., 0.05 ug/ul chocolate in BAX system, DuPont Qualicon). But Taq polymerase failed at this concentration (See e.g., FIG. 1, FIG. 2).

In a similar experiment, increasing amounts of green-tea extract (InVite), another polyphenol-containing substance, was added to PCR reactions.

Results showed that KT1, KT10, and FLAC2 were eight times more resistant to inhibition by green tea than Taq, tolerating up to 13.4 ng/ul tea extract (data not shown).

### Example 3

### PVP, PEC, and Casein Enhancer in Chocolate Samples

The following example demonstrates performance of a PCR enhancer containing PVP or casein in PCR reactions containing chocolate (and polyphenols, such as flavanoids contained therein).

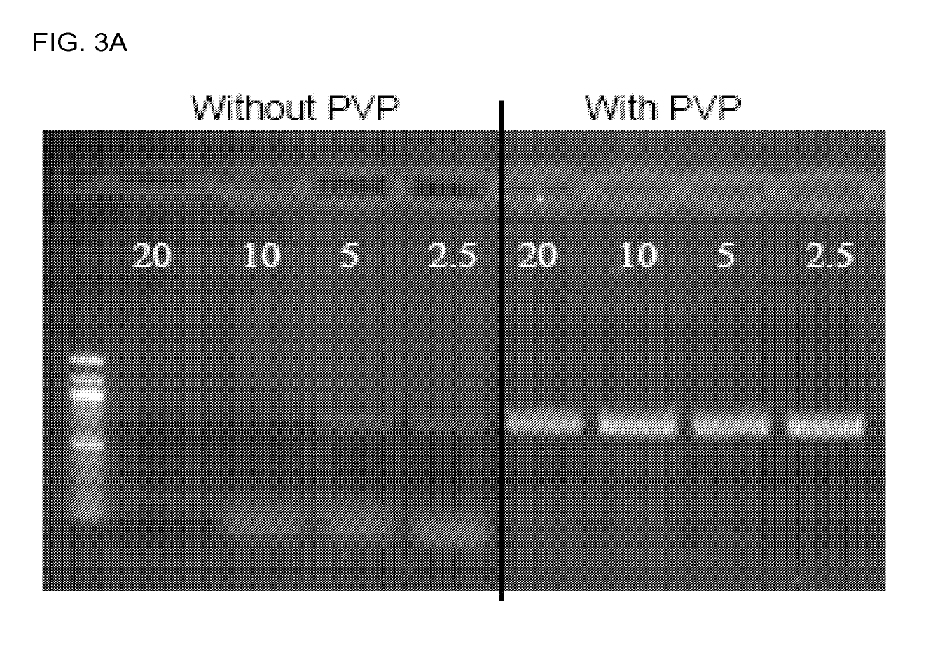

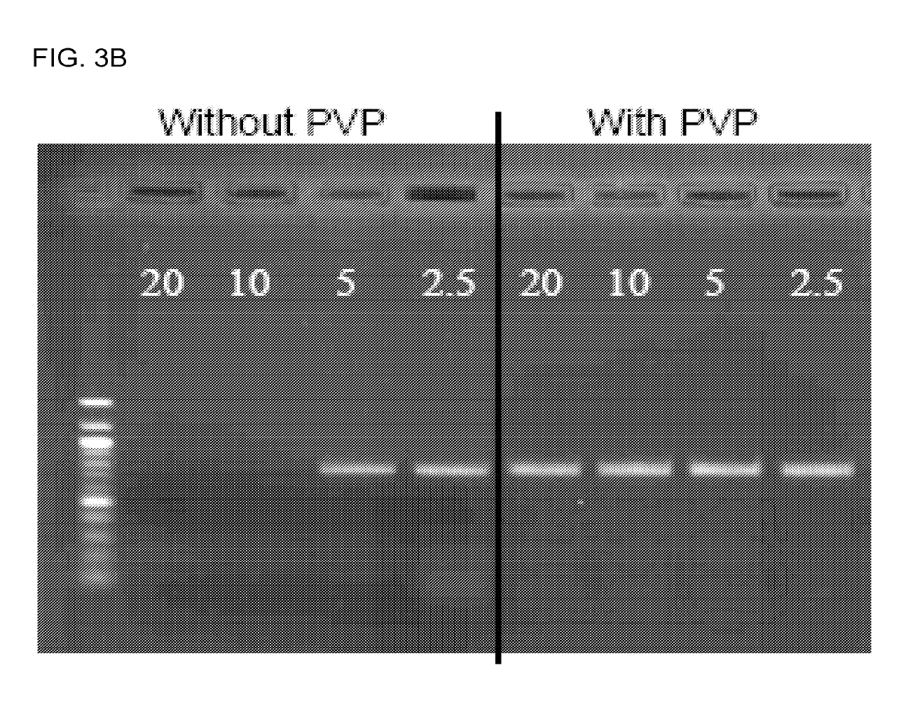

In a conventional PCR reaction, enriched cocoa was included in the reaction at concentrations of 20, 10, 5, and 2.5 μg per 50 μl reaction (comparable to, for example, a standard BAX reaction), with or without 8% PVP (see e.g., FIG. 3). Results showed that both OmniTaq and Omni Klentaq mutant polymerase enzymes in the presence of a casein enhancer composition allow robust amplification in 8× more chocolate than standard (see e.g., FIG. 3).

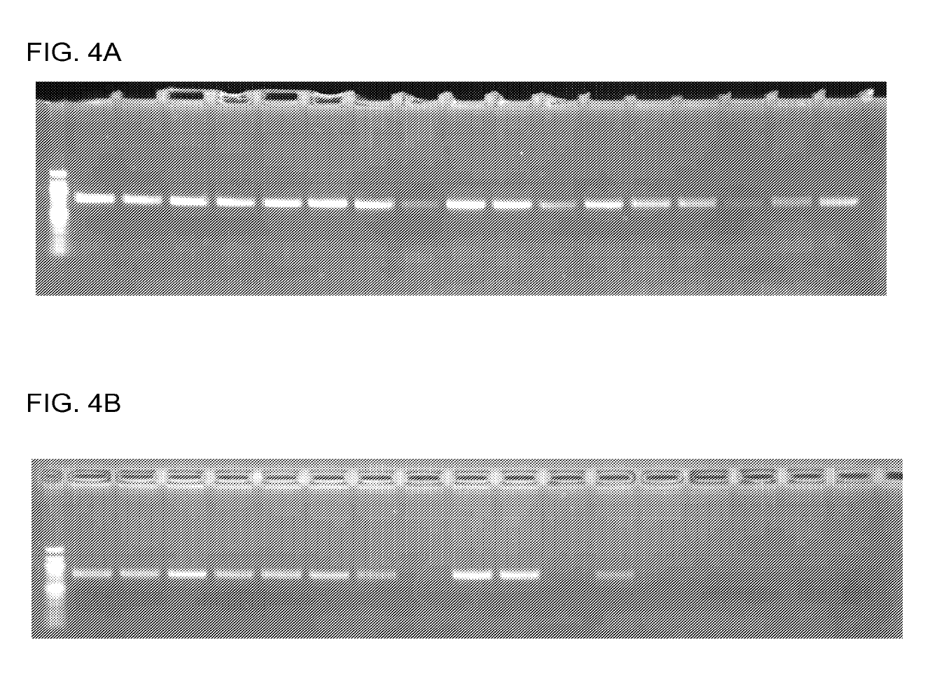

Conventional PCR was performed with an inhibition resistant mutant polymerase mixture of OKT:OT on a panel of chocolates spiked with Salmonella, each reaction containing 200 μg of chocolate (equivalent to 2 μl primary enrichment) with 8% PVP (see e.g., FIG. 4A) and without enhancer (see e.g., FIG. 4B). Results showed that, while inhibition resistant mutant polymerases were able to detect Salmonella in some samples, addition of 8% PVP allowed detection of Salmonella in all samples.

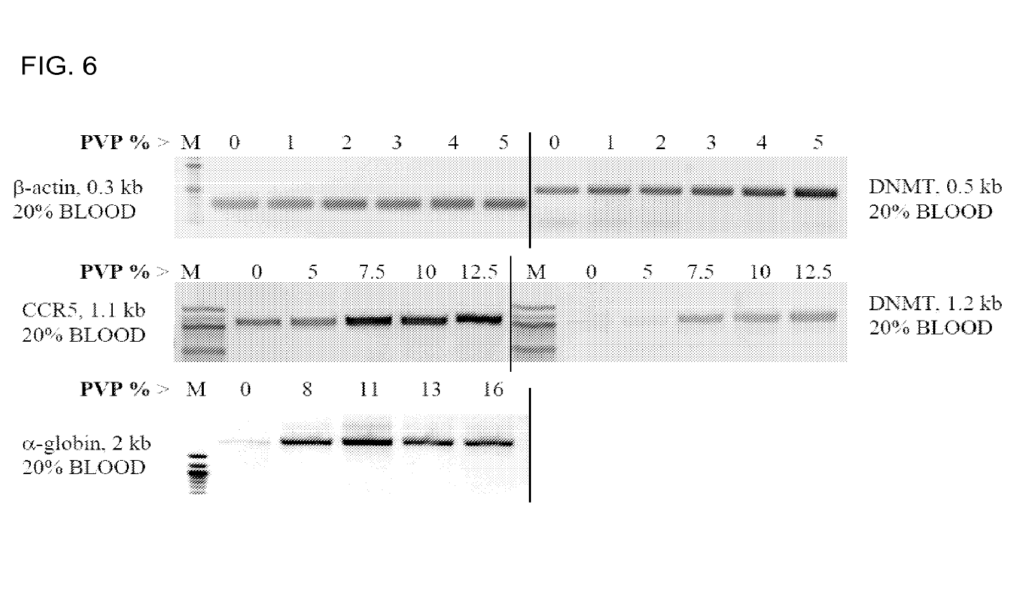

This example further shows that PVP acts as a potent facilitator of blood PCR. A strong enhancement of direct PCR amplification from whole blood was obtained with PVP. These results are surprising, as in the case of blood the main PCR inhibitors are hemoglobin, lactoferrin and an IgG fraction, rather than polyphenolic compounds, presuming a different mode of action of PVP, known to interact with polyphenols. This aspect of PVP is illustrated in an example where five endogenous human gene targets were amplified directly from 20% human whole blood with 0.5 μl Omni Klentaq per 50 μl reaction volume in the presence of increasing amounts of polyvinylpyrrolidone (PVP). The amplification was performed for 40 cycles, using a 6 min/95 degree initial heating step, and 3-6 minute extension time. The amplified products were analyzed in 1.5% ethidium bromide stained agarose gel. Lane M was the 100 bp DNA ladder. Results demonstrate that PVP significantly enhanced the amplification of targets in blood, more pronounced with relatively longer/more difficult targets, in which effect could be more pronounced if more than 12.5% PVP is used (see e.g., FIG. 6).

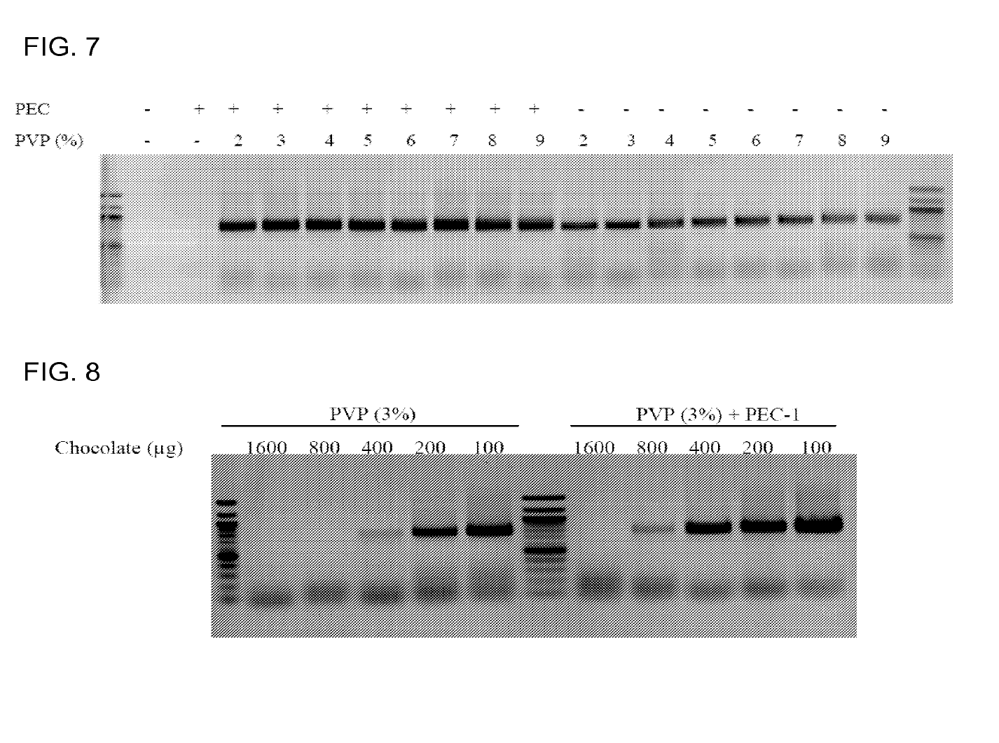

The following example demonstrates the detection of Salmonella DNA in chocolate samples containing polyphenolic compounds and other substances that inhibit PCR amplification with PVP+PEC. DNA was extracted from Salmonella cells and spiked in chocolate solution. A 783 bp Salmonella gene target was amplified in 50 μl PCR reactions containing 200 μg of chocolate and 1 ng DNA using 1 μl of 1×OmniTaq and 2%, 3%, 4%, 5%, 6%, 7%, 8%, 9% of PVP, respectively, with and without the PCR Enhancer Cocktail (PEC-1) which contained 0.6 M of D-(+)-trehalose, 0.2 M of L-carnitine and 0.25% of Briji-58. The PCR was performed for 40 cycles at 94° C. for initial denaturing, and then followed with 94° C. for 40 sec, 64° C. 40 sec and 70° C. 4 min. The PCR products were resolved in 1.5% agarose gel and stained with Ethidiom Bromide (see e.g., FIG. 7). The results showed that PVP alone was able to enhance the performance of OmniTaq, however, the combination of PVP and PEC demonstrated a synergistic effect (see e.g., FIG. 7).

Further experiments were performed to demonstrate the synergy of PEC and PVP on PCR in chocolate sample. Constant 1 ng of Salmonella DNA and 100 ng, 200 ng, 400 ng, 800 ng and 1600 ng of chocolate were added to 50 μl PCR reactions, respectively. A 783 bp Salmonella gene target was amplified using 1 μl of OmniTaq in the presence of PVP alone (3%, v/v) or PVP coupled with PEC-1. The PCR conditions were the same as in Example 1 except as otherwise indicated. The electrophoresis was done in 1.5% agarose gel and stained with Ethidium Bromide (see e.g., FIG. 8). Results indicate that the PVP+PEC-1 could tolerate 2 times more chocolate in the reaction than PVP alone (see e.g., FIG. 8).

### Example 4

### Improvement of Solid and Liquid Media for Salmonella Growth

This example demonstrates a reduction in inhibition in chocolate samples containing PVP.

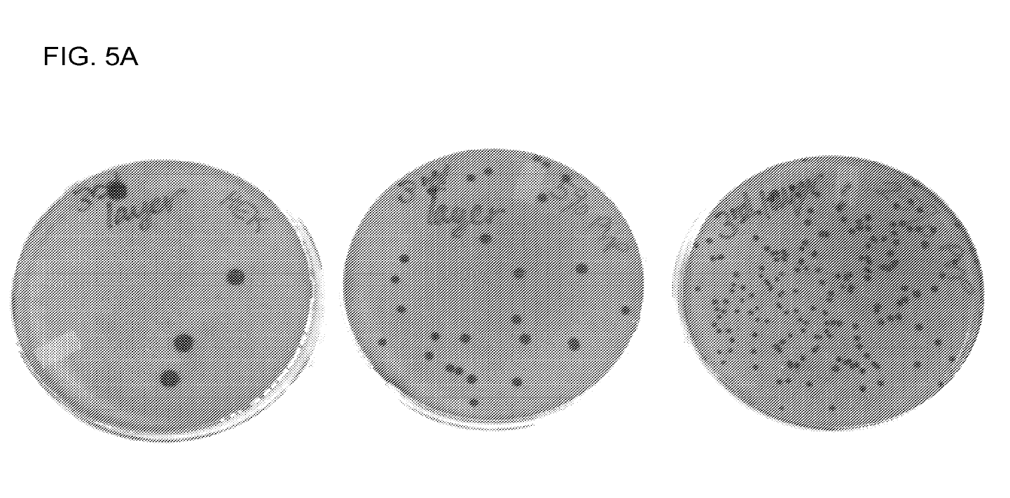

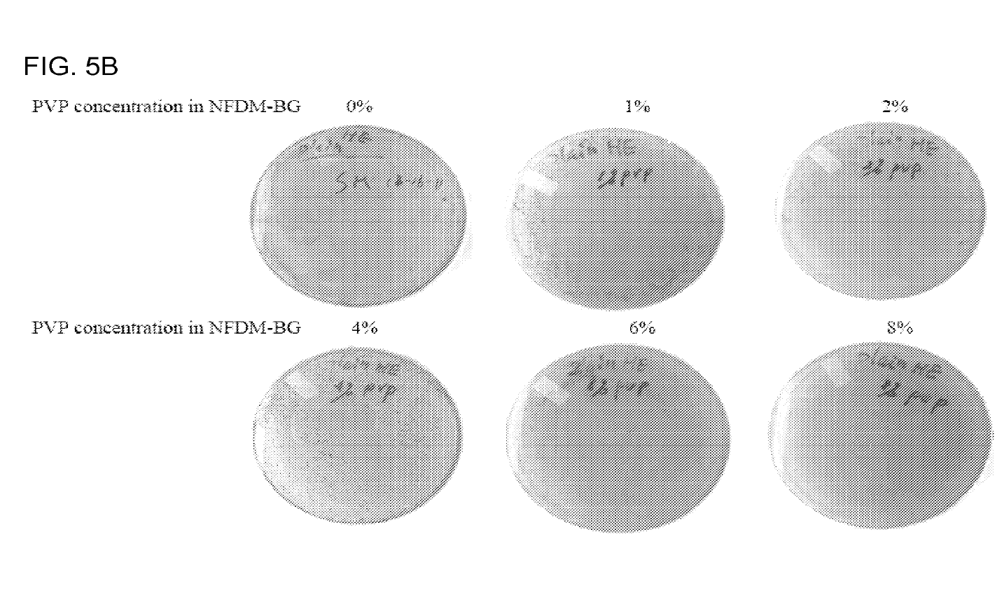

A chocolate sample was mixed 1:1 with water, spiked with salmonella cells, and spun for 5′ at 10,000 rpm. To determine the cells location, and also the inhibition of each layer on Salmonella growth, 50 μl of each layer was plated on HE plates. PVP was added to the plates at 5% and 15% to determine the effect of the chocolate inhibition and the HE plates without adding PVP as a control. The plates were incubated at 35° C. for 24 hours and the colonies were counted. Results demonstrate that PVP allowed many more colonies to grow by relieving the inhibition caused by chocolate. A clear reduction of inhibition was observed with the addition of PVP to the plates. Plates shown in FIG. 5A are from the 3rd (bottom) layer. The 1st (top) layer also showed reduction of inhibition on the HE-PVP plates with 2 colonies on the HE plate, 8 colonies on the 5% PVP, and 15 colonies on the 25% PVP. The 2nd (middle) layer, perhaps the least inhibitory, had ˜400 colonies on both the 5% and 25% PVP plates (No data for the HE plate).

Further results in liquid media showed that PVP at 1%-4% was able to relieve inhibition of chocolate or stimulate Salmonella growth. The PVP was added to NFDM-BG media (Non-Fat Dry Milk with Brilliant Green) at 0%, 1%, 2%, 4%, 6% and 8%, respectively final concentrations. A chocolate was mixed with these media at 1:10 ratio (w/v) and Salmonella cells were inoculated inside and enriched at 350 C for 24 hours. After incubation, 100 μl of middle layer from these samples were plated on HE plates and incubated at 350 C for 24 hours. When PVP concentration is higher than 4%, such as 6% and 8%, inhibition of Salmonella growth was observed (see e.g., FIG. 5B).

### Example 5

### PVP and SYBR

The following example demonstrates performance of a PCR enhancer containing casein in real time PCR with SYBR Green detection. As shown below, 8% PVP overcomes SYBR inhibition, allowing for higher amounts of SYBR in the reactions.

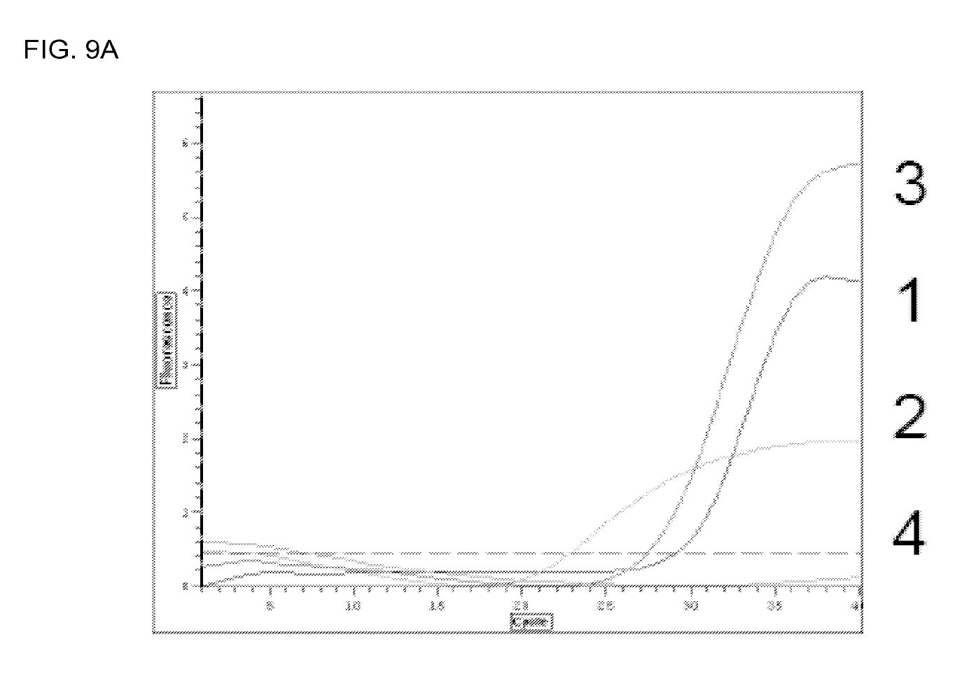

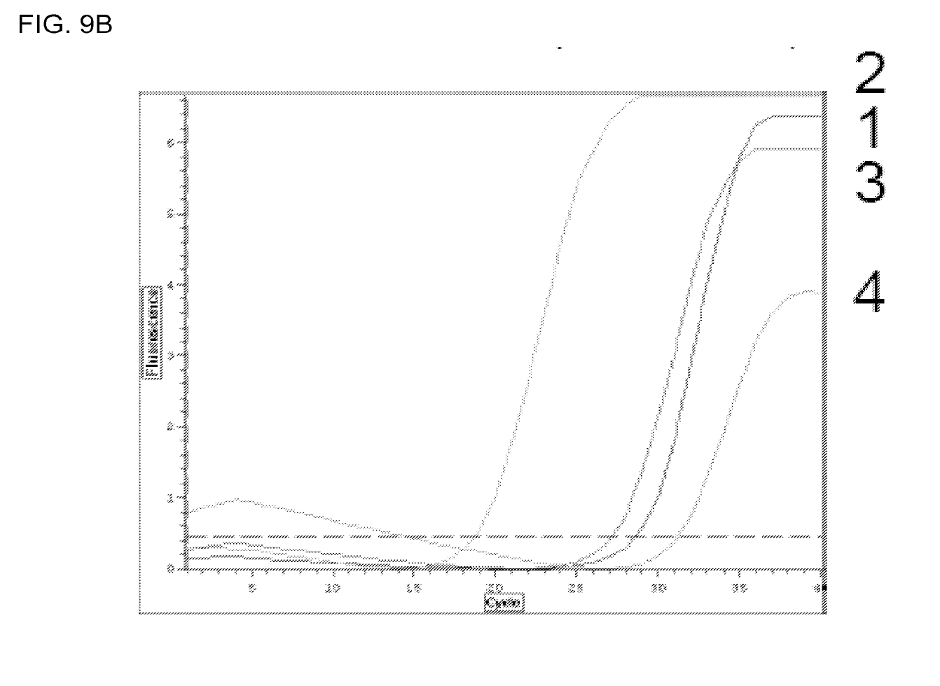

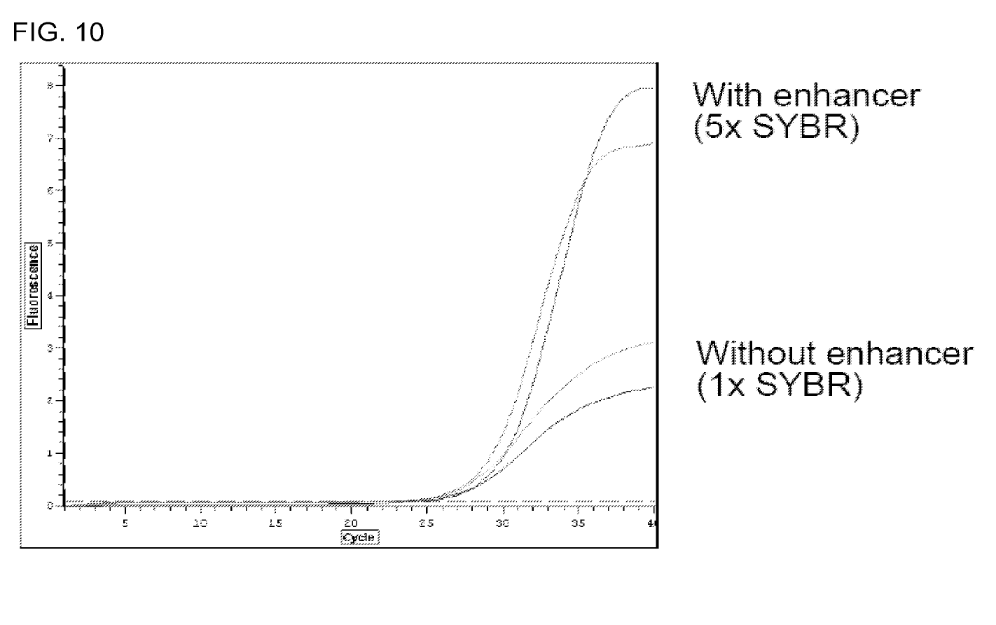

Samples containing chocolate were spiked with Salmonella and enriched. Amplification reactions contained 2 μl per reaction. DNA detection was according to real-time PCR without enhancer and 1×SYBR (see e.g., FIG. 9A), real-time PCR with 8% PVP enhancer and 5×SYBR (see e.g., FIG. 9B). Labels 1-4 designate four different chocolates. Results showed that with addition of 8% PVP, Salmonella could be detected by real-time PCR in all samples directly from the primary culture (see e.g., FIG. 9B). Results also showed that Salmonella could be detected by BAX directly from the primary culture.

To show the effect of PVP enhancer on SYBR concentration, 125 μg of cheese were spiked with 30 cells of Salmonella and real-time PCR conducted with either 1×SYBR or 5×SYBR. Results showed enhanced detection of Salmonella in real-time PCR reactions using 5×SYBR (see e.g., FIG. 10). It is noted that without the 8% PVP, 5×SYBR was observed to kill the reaction (data not shown).

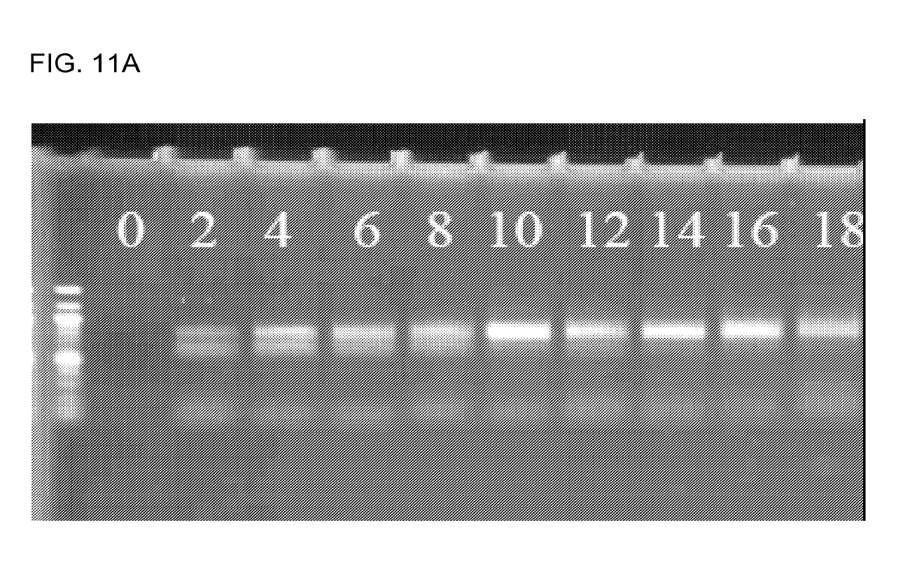

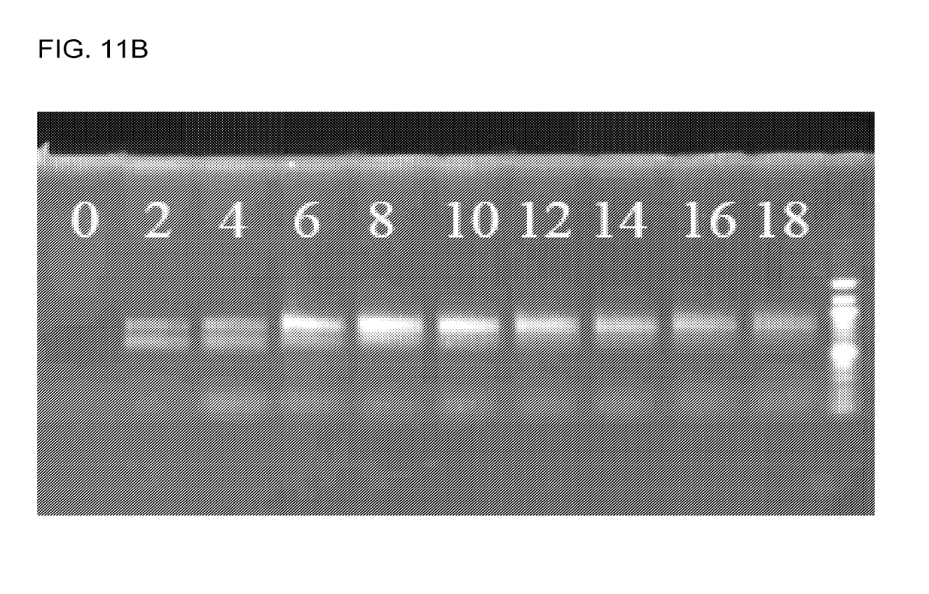

To optimize PVP concentration, PCR reactions with OmniTaq (See e.g., FIG. 11A) or Omni Klentaq (see e.g., FIG. 11B) were run with samples containing chocolate spiked with Salmonella, 5×SYBR, and PVP at a concentration of 0, 2%, 4%, 6%, 8%, 10%, 12%, 14%, 16%, or 18%. Results showed that at low concentrations of PVP, a double band is present, which is an indication of SYBR inhibition (see e.g., FIG. 11). Results also showed that a lower realtime signal was observed at higher concentrations of PVP (see e.g., FIG. 11). Thus an optimal PVP concentration of 8% was determined.

### Example 6

### PVP and Microflora

The following example shows that PEC and 8% PVP can overcome inhibition by competing microflora.

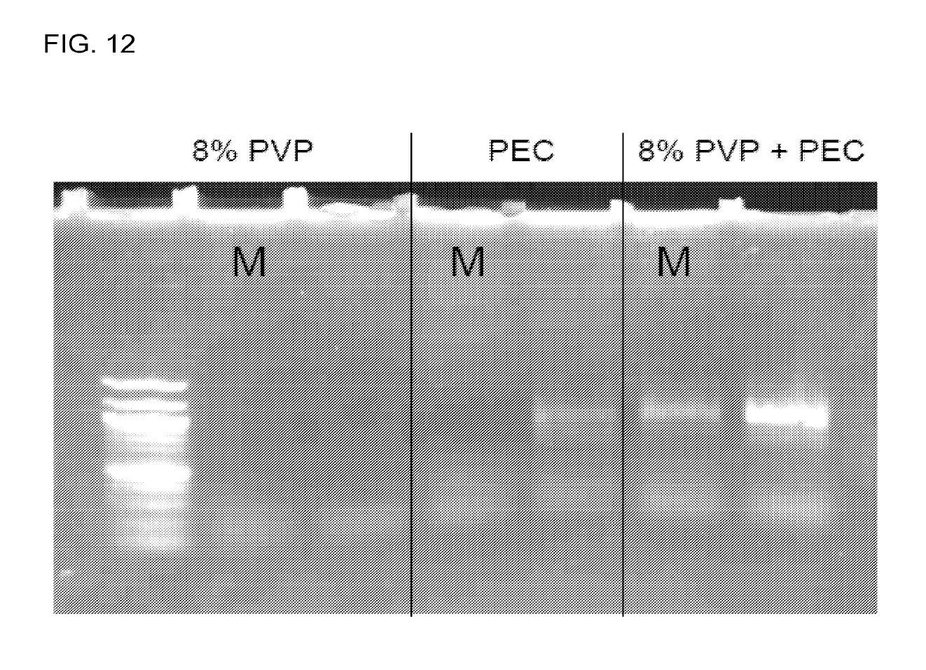

A chocolate sample underwent primary enrichment without Salmonella to grow up competing microflora. The enriched chocolate samples were spiked with Salmonella DNA and detection of samples containing 400 μg of chocolate was conducted according to real-time PCR with 8% PVP, PEC, or 8% PVP and PEC. Results showed robust detection of Salmonella DNA in the presence of both PEC and 8% PVP (see e.g., FIG. 12).

### Example 7

### Comparison of Milk, BSA, and Casein

The following example compares the effect of milk, BSA, and casein on the inhibition of polyphenols in samples containing chocolate.

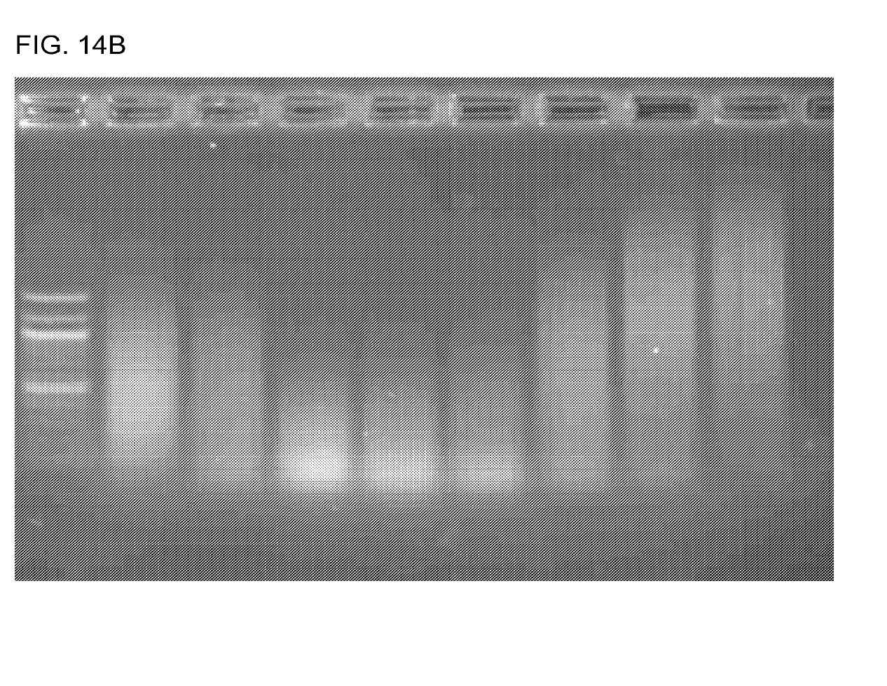

Samples contained 200 μg of chocolate and 0.2% milk, 0.16% casein, 0.16% BSA, or 0.16% casiene and PEC. Each subsequent lane of FIG. 14 doubles the protein concentration. All reactions contained template (salmonella cells), salmonella-specific primers, dntps, and OmniTaq-LA polymerase. Reactions were cycled for 95° for 8′, then 33 times [95° 50″, 66° 1′, 68° 6′].

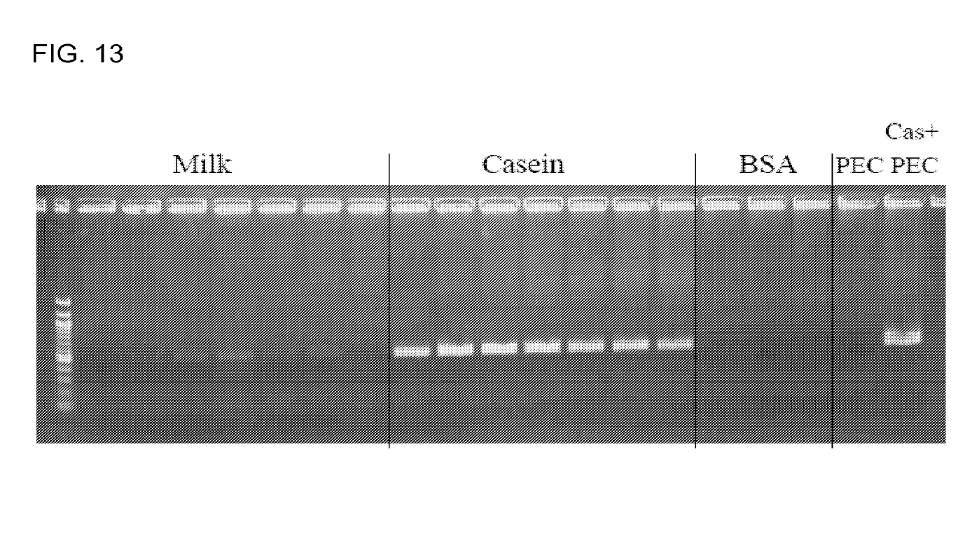

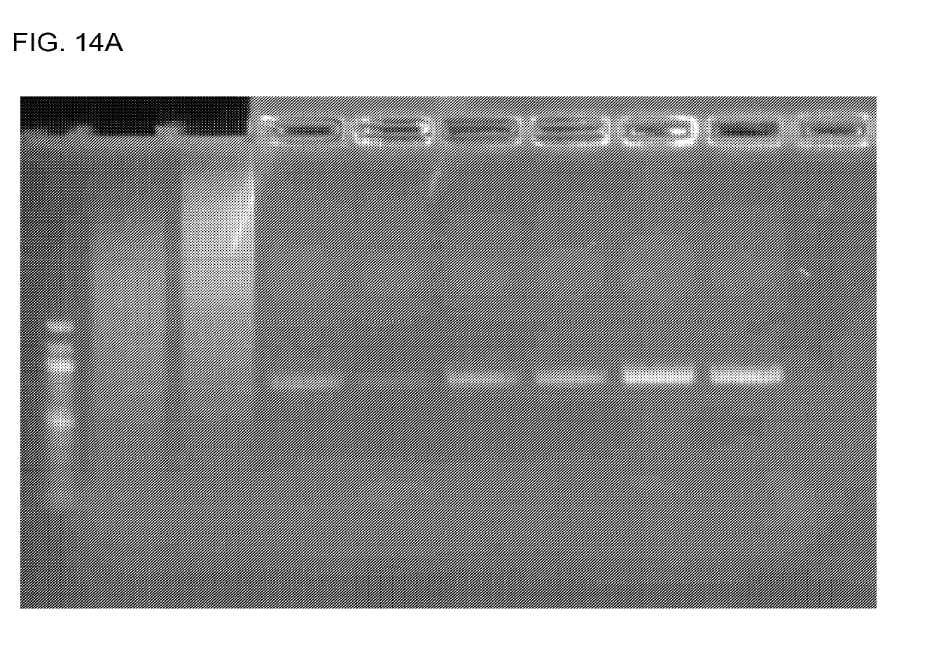

Results showed casein enhancer provided for salmonella detection. In contrast, samples containing milk, BSA, or PEC, salmonella detection was not robust (see e.g., FIG. 13).

### Example 8

### Potato Peel and Casein or PVP

The following example demonstrates detection of Salmonella in potato peel samples.

A potato peel was crushed in water and decreasing two-fold dilutions were added to PCR reactions for inhibition. Detection of Salmonella was attempted according to PCR protocols in the presence of 0.4% casein or 8% PVP.

Results showed that without enhancer or in the presence of PEC (both not shown), only the lowest two dilutions were amplifiable. Results also showed that 0.4% casein allowed for the tolerance of 16 times this amount (see e.g., FIG. 14). PVP was not observed to provide successful detection at any concentration (see e.g., FIG. 14).

### Example 9

### Soil and PVP or Casein

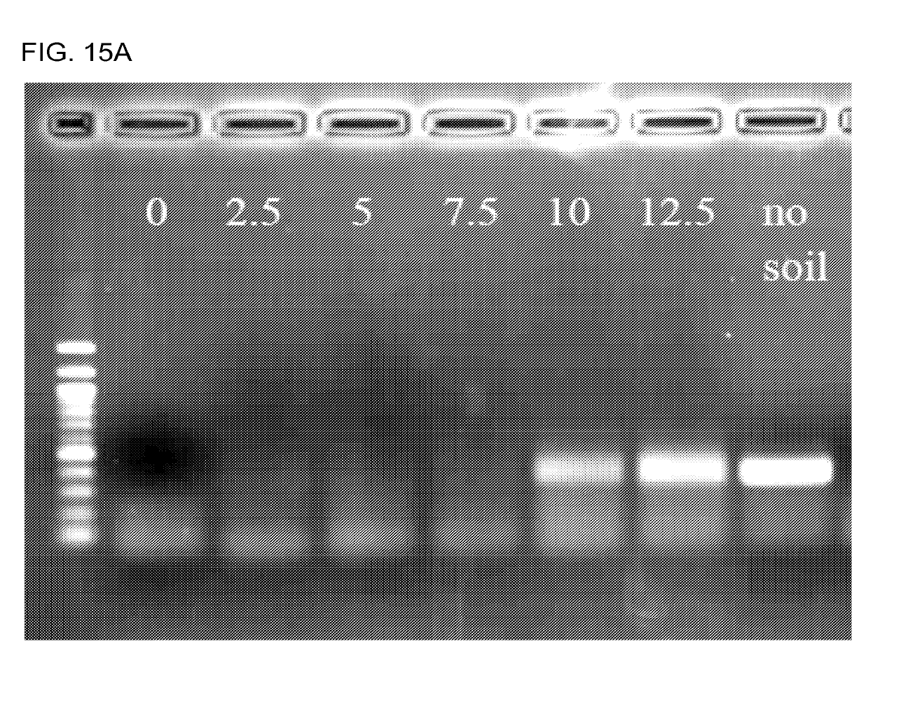

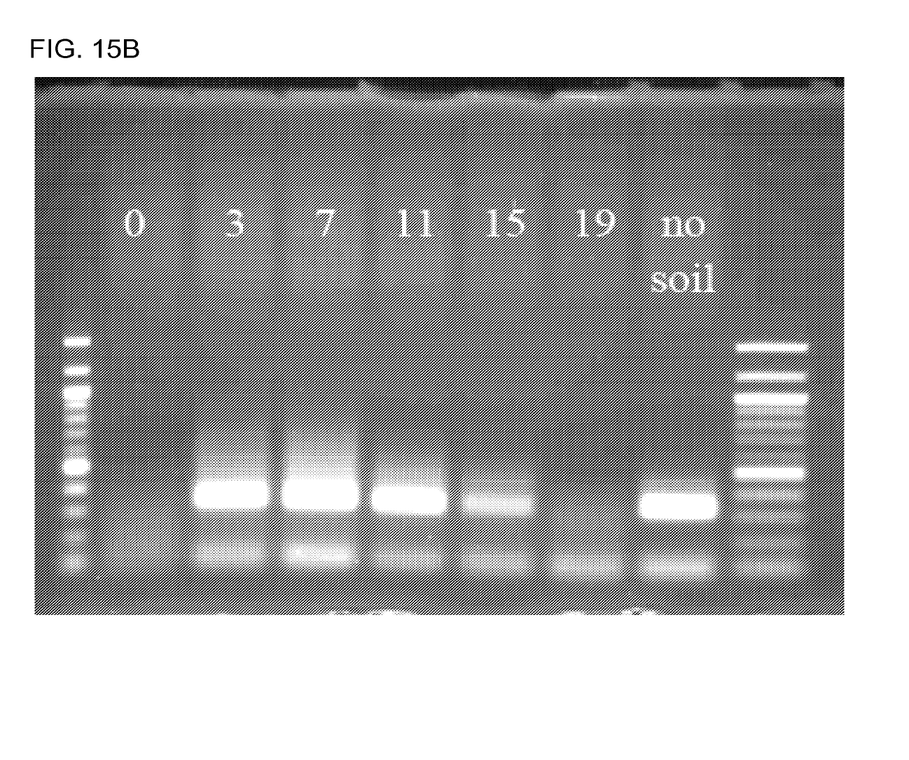

The following example demonstrates detection of soybean cyst nematode DNA in soil samples containing humic acid, a polyphenolic PCR inhibitor. Reactions contained a fixed amount of soil and 0%, 2.5%, 5%, 7.5%, 10%, or 12.5% PVP (see e.g., FIG. 15A). Results showed that lower concentrations (e.g., 0-7.5%) of PVP were not effective to provide for detection of soybean cyst nematode DNA in soil samples but PVP at 10% or 12.5% provided for detection of soybean cyst nematode DNA in soil samples (see e.g., FIG. 15A).

Reactions contained 0.4% casein and 0%, 3%, 7%, 11%, 15%, or 19% soil (see e.g., FIG. 15B). Results showed that 0.4% casein was effective at detection of soybean cyst nematode DNA in soil samples at all but the highest concentrations of soil (e.g., 3%-15%, but not 19%), with the strongest enhancement observed at about 3% to about 11% soil, particularly 7% soil (see e.g., FIG. 15B).

Further experiments also show PVP or casein overcome PCR inhibition by humic acid. Environmental/soil specimens represent another class of challenging PCR samples due to the strong PCR inhibition by the humic acid present in soil. This example demonstrates that both PVP alone and casein alone can improve the resistance to humic acid inhibition in PCR. A 250 bp test target of Lambda DNA was amplified from 1 ng DNA for 35 cycles with 0.2 μl Omni Klentaq in the presence of 40 ng, 13.3 ng, 4.4 ng, 1.5 ng and 0.5 ng humic acid (lanes 1-5). Control reactions (lanes 6) contained no humic acid. The reaction sets were performed without additives, with 9% PVP, and with 0.4% casein. Lanes M was the DNA ladder. The amplified products were resolved in a 2% ethidium bromide stained agarose gel.

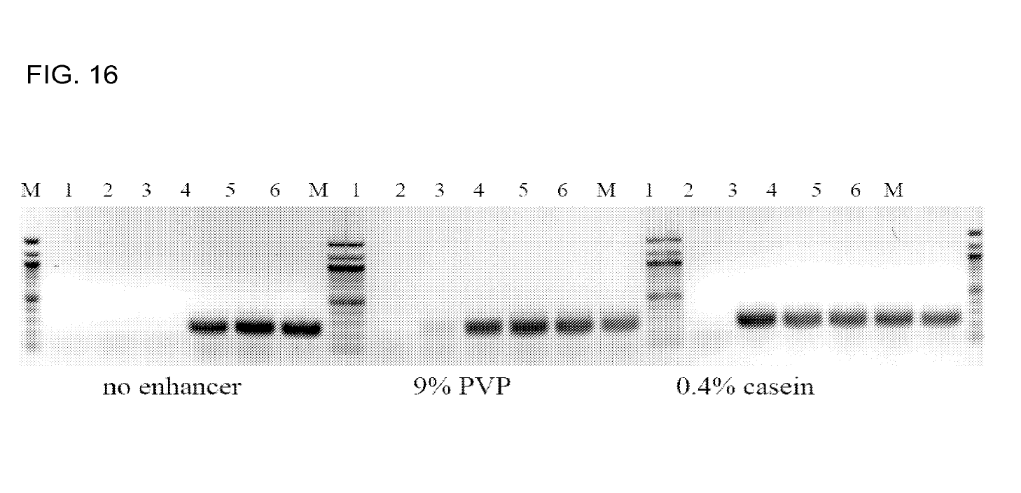

Results indicate that PVP and casein increased the tolerance to the PCR inhibitor about 3-fold and 9-fold, respectively (see e.g., FIG. 16).

In a separate experiment (results not shown), similar results were obtained with equivalent amounts of plain Taq DNA polymerase (New England Biolabs).

### Example 10

### Dried Blood and PEC or PEC+PVP

The following example shows direct amplification of HCV gene from a dried blood spot on a FTA card and a paper card.

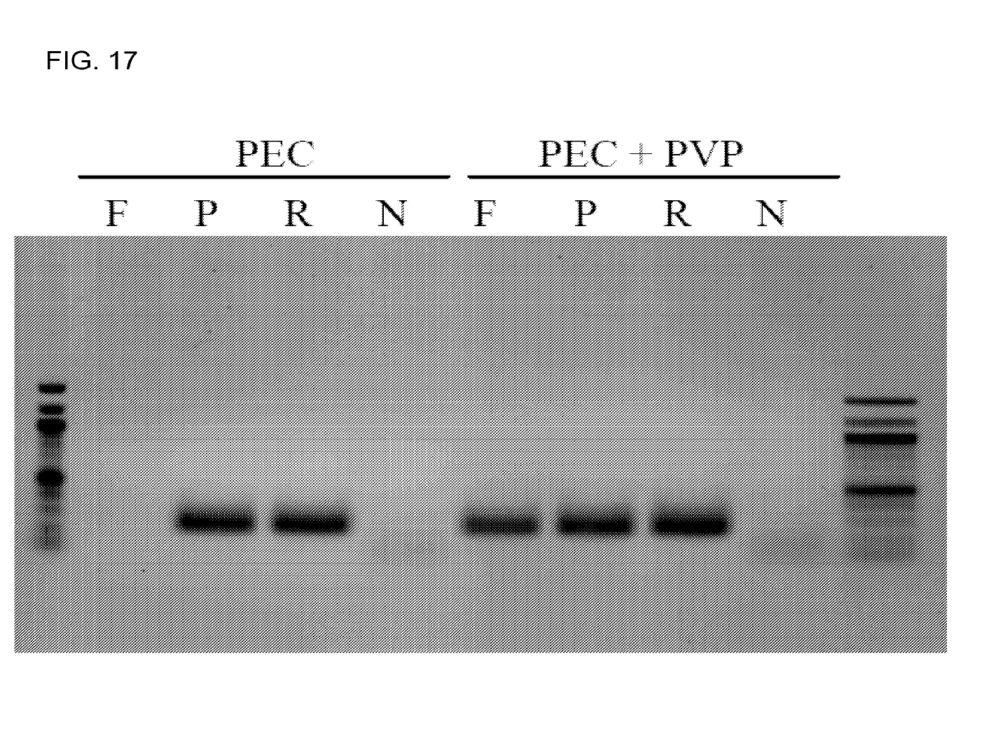

HCV positive whole blood (˜50 μl) was dripped to FTA or general paper card and a 1.2 mm piece was punched with a puncher and put into the PCR reaction. A 224 bp HCV gene was directly amplified from the dried blood spot using a 1 μl mixture of OmniTaq and Omni Klentaq blended with 0.5 μl of SuperScript III, in the presence of PEC-1 or PEC-P (which is PEC-1 with an additional 2% PVP). The same amount of purified RNA was included as a positive control and the template was withheld for the negative control. The one-step RT-PCR was performed at 55° C. for 30 min in the reverse transcription step and immediately inactivated in reverse transcriptase at 94° C. for 5 min followed by 40 cycles at 94° C. 30 sec, 54° C. 40 sec and 70° C. 1.5 min. For each reaction, 10 μl of PCR product was loaded in 1.5% agarose gel for electrophoresis (see e.g., FIG. 17)

The results showed that the RT-PCR system, consisting of a blend of OmniTaq, Omni Klentaq, and SuperScript III, efficiently amplified the RNA target from a dried blood spot on the general paper card in the presence of PEC-1, but was not able to amplify the target on the FTA card. When a combination of PEC-1 and PVP was used, the RT-PCR system was able to amplify the RNA target from both the general paper card and the FTA card. The same amount of purified RNA was included as a positive control and the template was withheld for the negative control (see e.g., FIG. 17).

### Example 11

### Soybean or Tomato Leaf and PVP+PEC

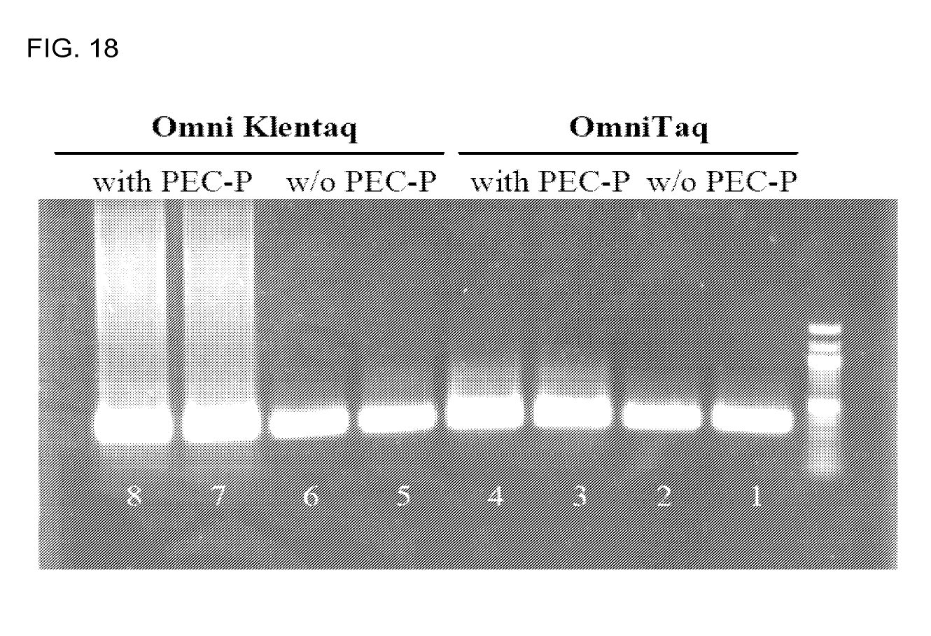

The following example shows direct amplification of ribosomal 16s gene from plants. A 1.2 mm piece of soybean or tomato leaf was punched and applied in a 25 μl PCR reaction containing 1× reaction buffer, 200 μM dNTP and 200 nM of forward and reverse primers. A 365 bp gene was directly amplified from these samples using 1 μl of OmniTaq or Omni Klentaq in the presence or absence of PEC-P. The PCR was run at 94° C. 10 min for the initial pre-heating and then followed by 40 cycles at 94° C. 40 sec, 54° C. 45 sec and 70° C. for 2 min. The amplified products were resolved in 1.5% agarose gel and stained with Ethidium Bromide(see e.g., FIG. 18). The results showed that the Taq mutants were able to directly amplify this target without DNA extraction prior to PCR. The yields were higher in the presence of PEC-P (see e.g., FIG. 18).

### Example 12

### Tobacco Leaf and PEC

The following example shows the enhancing effect of PEC-PVP or PEC-casein cocktail on DNA amplification in the presence of crude tobacco leaf extract.

Plants contain various potent PCR inhibitors, predominantly polyphenols, which may compromise the PCR-based tests and studies. Therefore, extensive DNA extraction and purification protocols are typically required prior to PCR.

This example illustrates the effect of PVP combined with PEC (4% PVP final concentration) in overcoming plant tissue PCR inhibitors, allowing direct amplification of DNA from crude plant extracts, without any DNA purification steps involved.

A crude extract was prepared by homogenizing tobacco leaves in Omni Klentaq PCR buffer with glass beads in a bead beater for 10 min, followed by 20 min heating at 75 deg and a short spin.

A 320 bp test target of the human b-actin gene was amplified from 1 ng human DNA with 0.5 μl Omni Klentaq enzyme for 40 cycles in the presence of 1, 2, 4, 8, 12 and 16 μl of a crude extract from tobacco leaves (lanes 2-7). Control reactions (lanes 1) contained no plant tissue extract. This set of reactions was preformed with no additives (left top panel), in the presence of PEC-1 (top right panel), or in the presence of PEC-1 supplemented with PVP to a final concentration of 4% in the reaction (bottom panel). Lanes M, DNA ladders. The amplified products were analyzed in a 1.5% ethidium bromide stained agarose gel. The PEC-PVP combination clearly outperformed the standard (plain) PEC, allowing amplification in all plant extract concentrations tested.

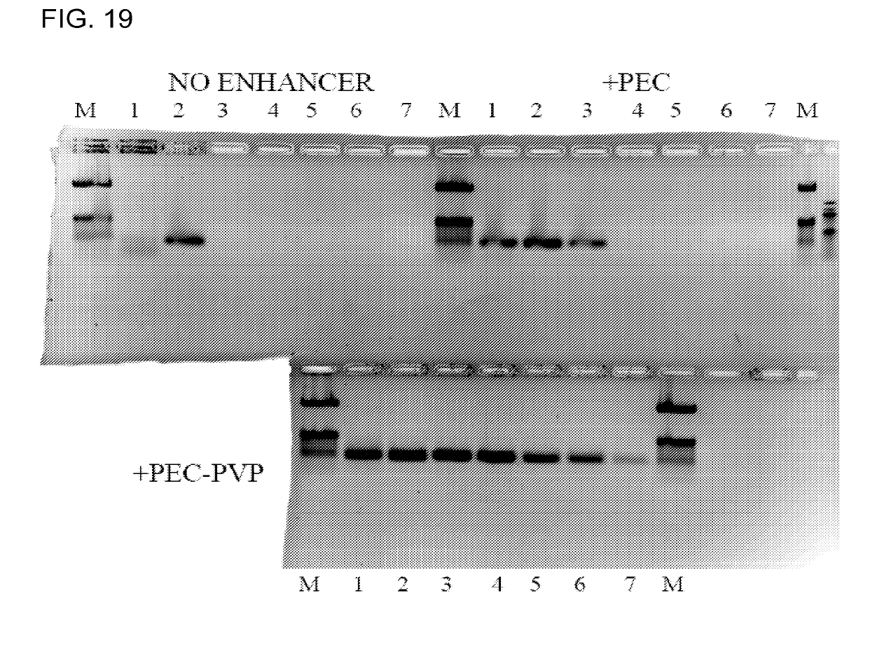

Results show that the PEC-PVP combined enhancer cocktail helps significantly overcoming the plant tissue PCR inhibitors (see e.g., FIG. 19).

Further studies examined the effects of casein in overcoming plant tissue PCR inhibitors. For example, a human DNA test target was amplified by both Omni Klentaq and plain Taq in the presence of increasing amounts of tobacco leaf crude extract, up to 16% extract in the final reaction volume. Results showed that tolerance of both Omni Klentaq and a plain Taq (NEB) to the plant inhibitors was increased at least 4-8 times in the presence of 0.6% Casein (data not shown).

### Example 13

### Other Candidate Enhancers

The following example demonstrates that various additives are not effective at for decreasing inhibitory effects of polyphenols in samples. Methods are according to those described above unless indicated otherwise.

Experiments using milk and BSA showed significantly less amplification in a polyphenol containing sample, as compared to casein.

Similarly, gelatin, polyproline, cyclodextrin, and isinglass were shown to not be as effective as casein for decreasing inhibitory effects of polyphenols in samples. Gelatin and polyproline are similar protein candidates. Polyproline was thought to be a proline-rich protein that may precipitate polyphenolic compounds (see Luck et al. 1994 Phytochemistry 37(2), 357-371), but such results were not observed. Cyclodextrin was thought to potentially behave similar to casein micelles encapsulating inhibitory compounds, but such results were not observed. Also, micelle-formation buffers to assist casein in forming better micelles were show to be ineffective.

### Example 14

### Human Short Tandem Repeats (STR) Samples Containing Bile Salts, Indigo Dye, and Tannins

The following example demonstrates the benefit of using PVP in combination with PEC in forensics. Short Tandem Repeat (STR) genotyping of human DNA was performed in the presence of challenging amounts of some potent PCR inhibitors found in forensic specimens, such as bile salts, indigo dyes or tannins (amounts specified in the legends of the corresponding figures) which may compromise forensic test results. In these conditions full STR profiles were generated with the PowerPlex16 HS primers set only when using OmniTaq or CesiumTaq enzyme supplemented with a PEC-4% PVP cocktail, performing in the standard genotyping protocol and cycling conditions (Promega). Like other challenging cases, PEC alone was not as efficient as the PEC-PVP combination (not illustrated), and two top commercial kits, Identifier Plus and PowerPlex-16 HS, failed to produce STR profiles.

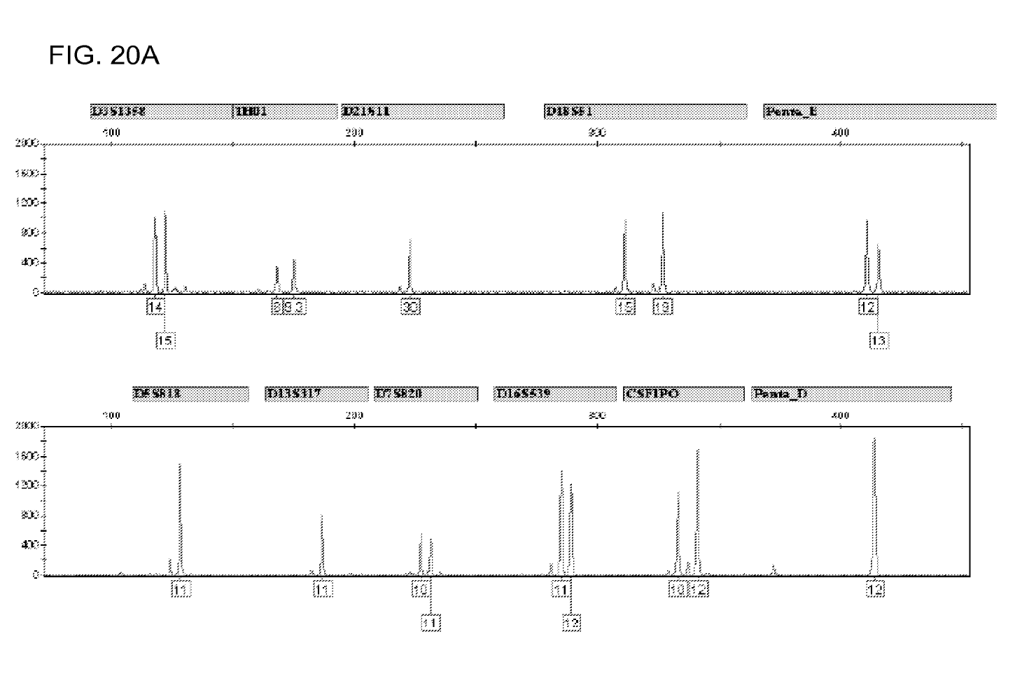

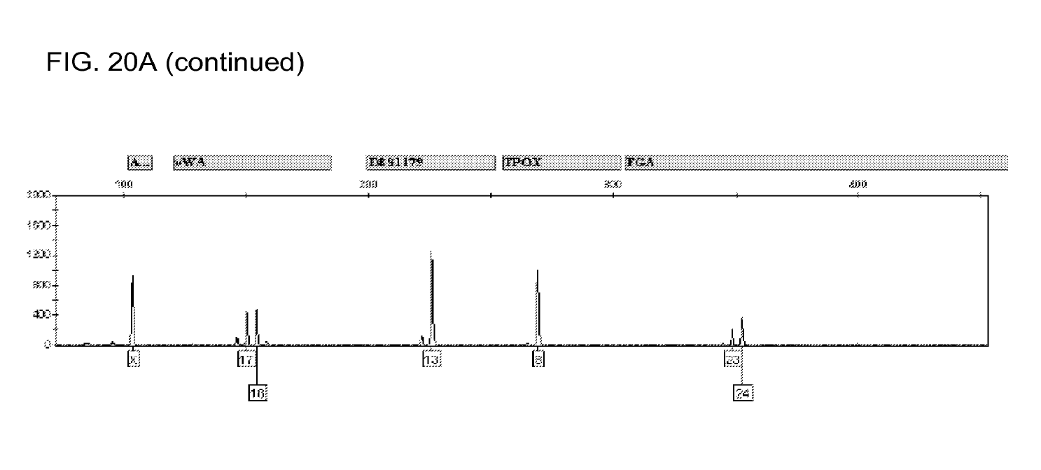

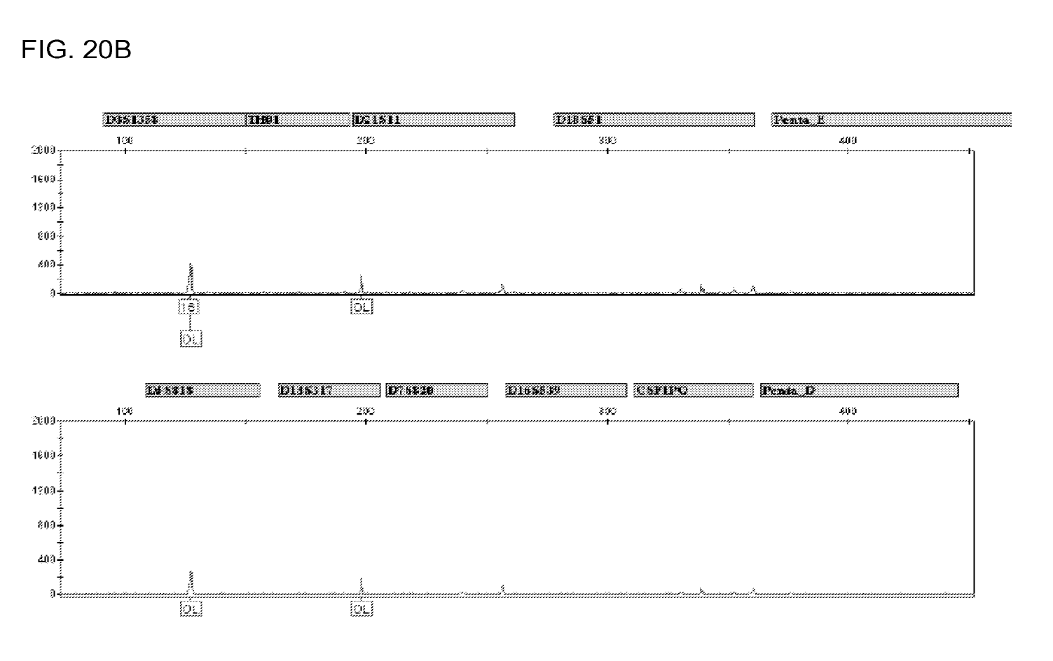

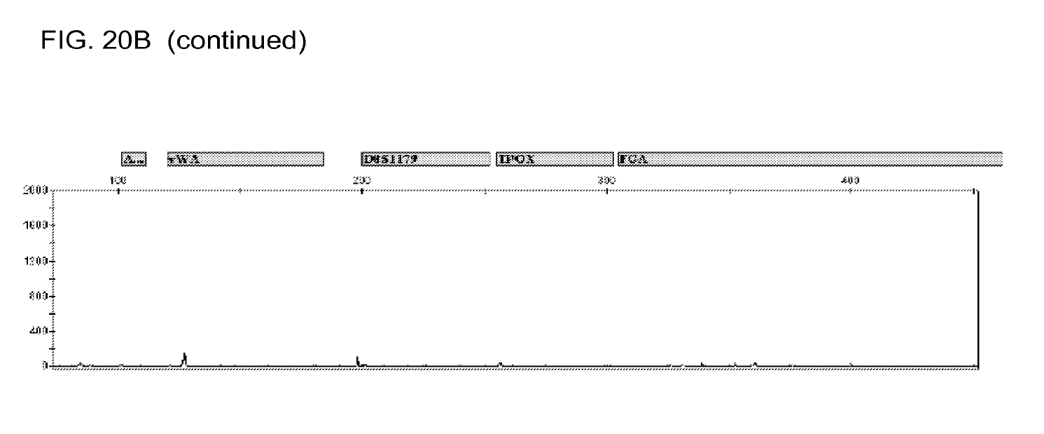

The following example shows the direct STR genotyping of crude samples containing bile salts. The results show that a full profile can be generated with the enzyme and enhancer cocktail (combining with PEC-P) where the PP16HS Promega kit failed to obtain a profile (see e.g., FIG. 20A and FIG. 20B).

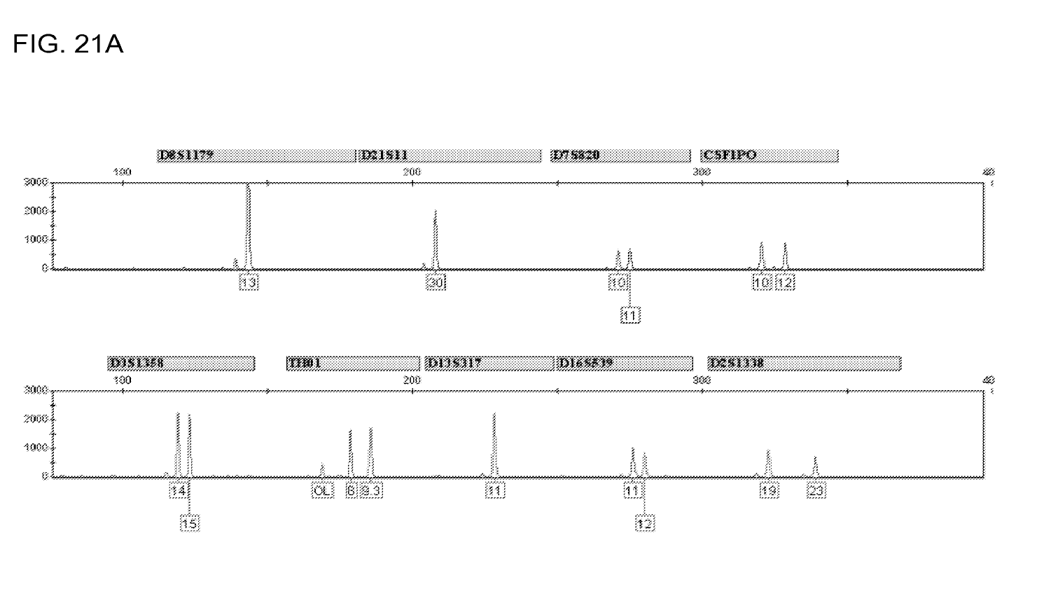

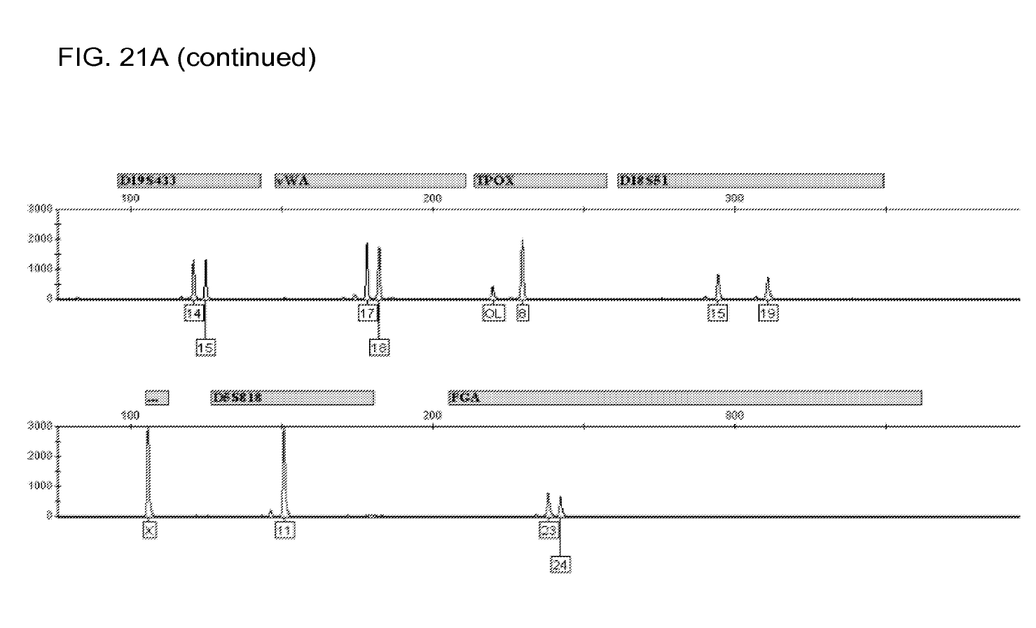

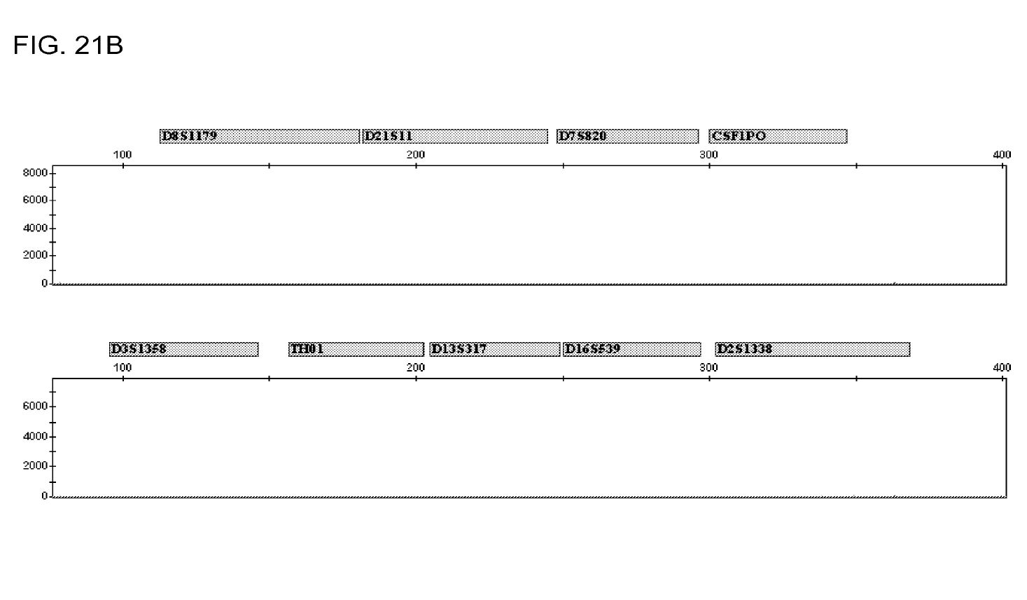

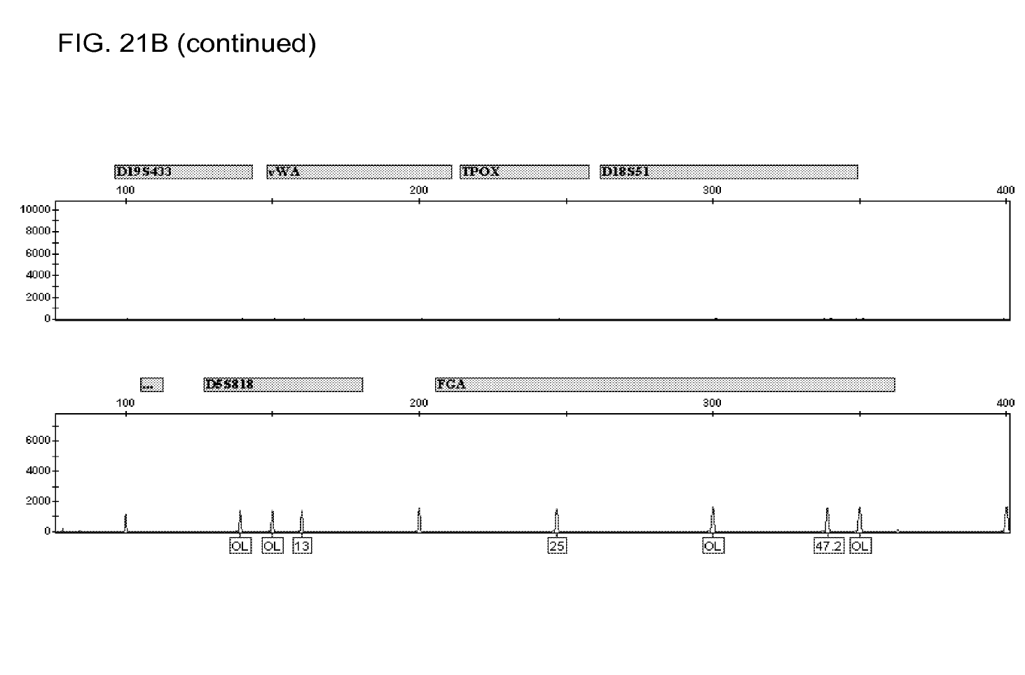

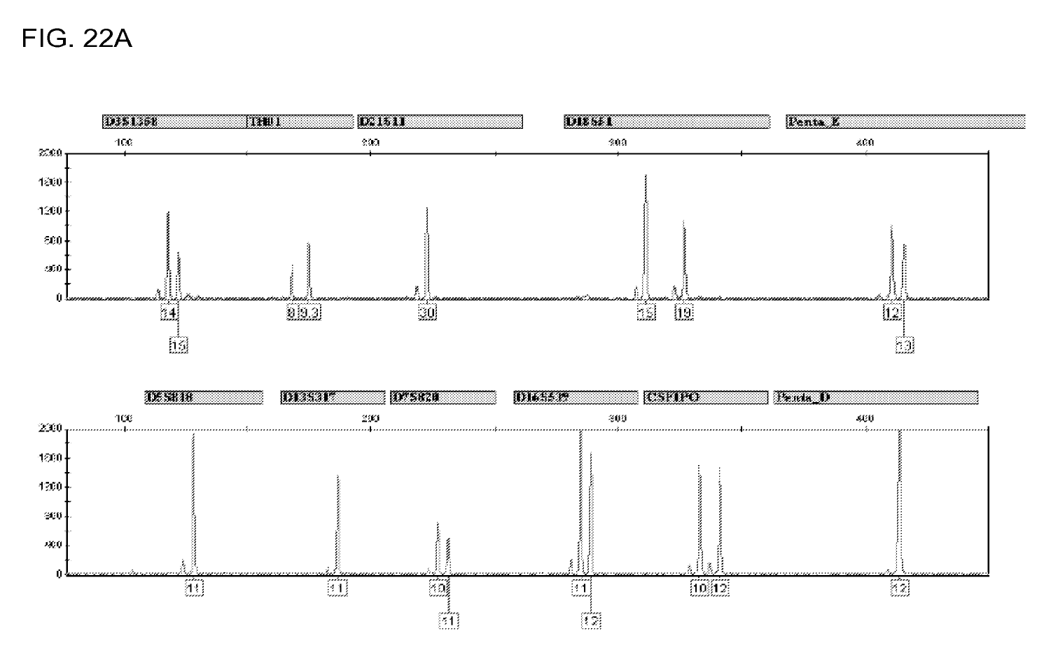

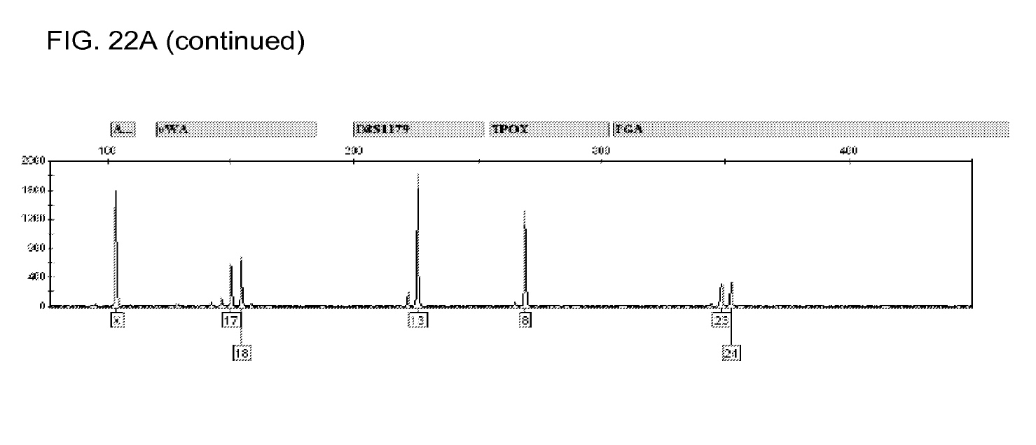

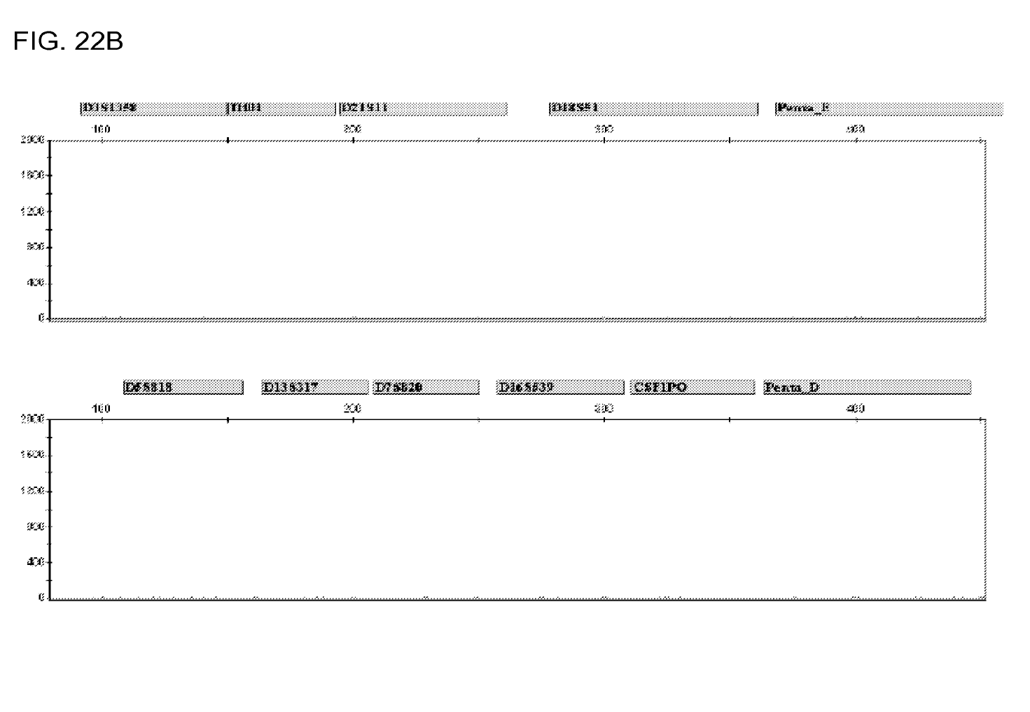

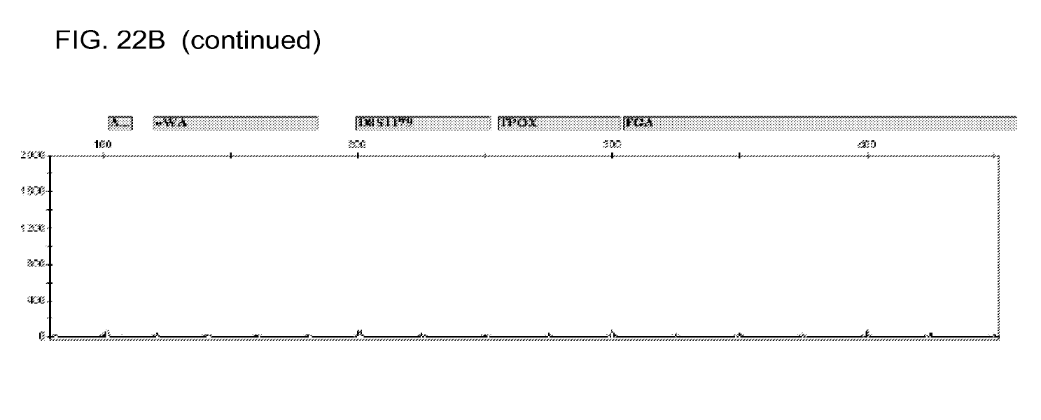

The following example shows the direct STR genotyping of crude human DNA samples containing indigo dye. The results show that the combination of the novel mutant enzyme and PEC-PVP can tolerate the inhibitory dye and generate a full genotyping profile, while a weak partial profile with spurious peaks were produced with the ID-PLUS kit (see e.g., FIG. 21A and FIG. 21B). Further, the results show that the novel mutant enzyme, supplemented with the enhancer cocktail, can tolerate the inhibitory dye and generate a full genotyping profile, while no profile was produced with the PP16 HS kit (see e.g., FIG. 22A and FIG. 22B).

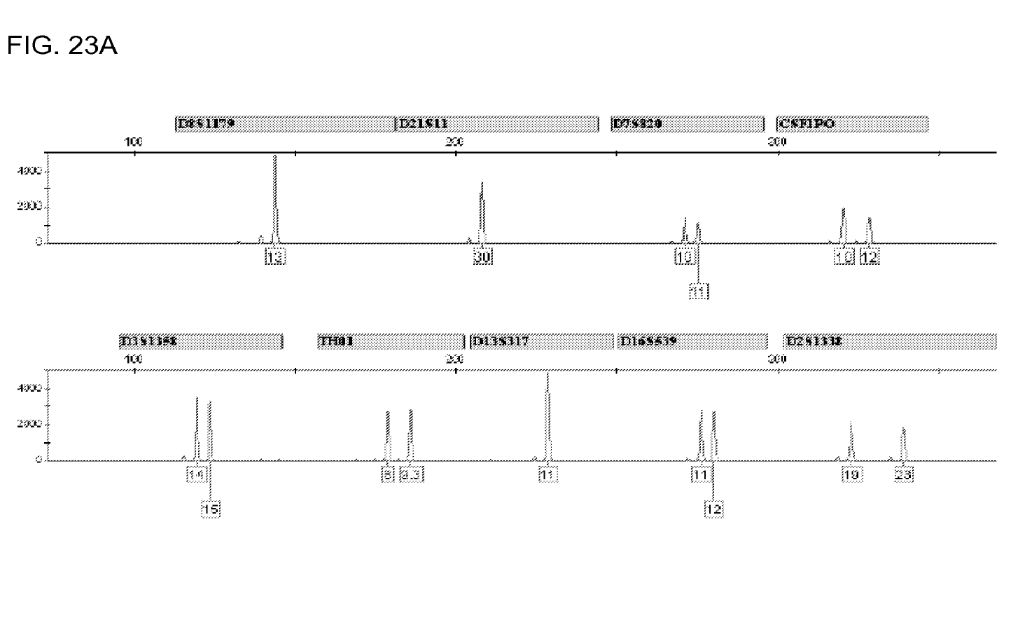

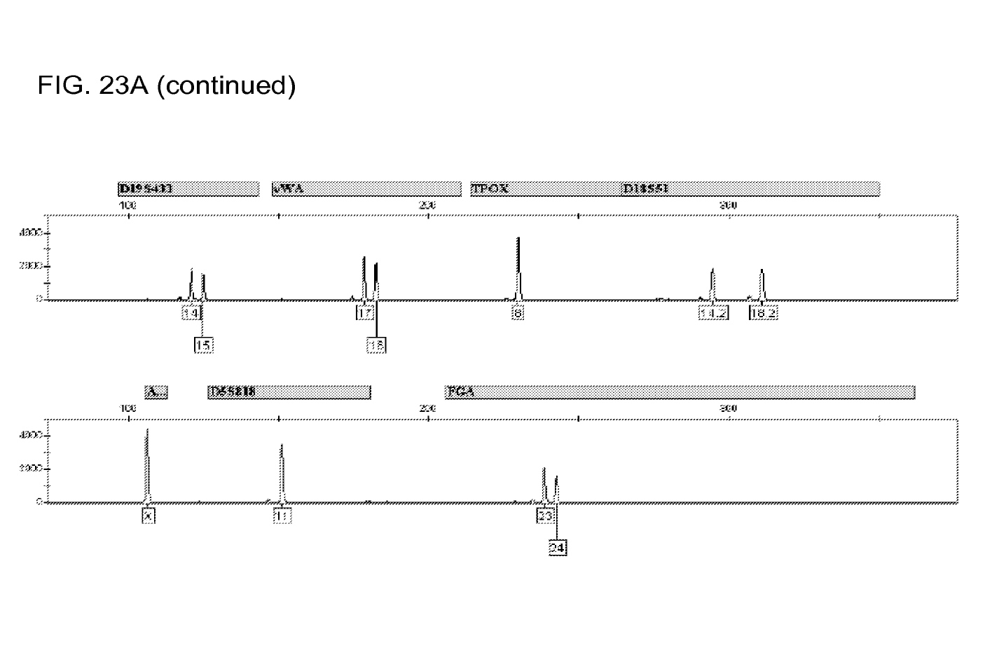

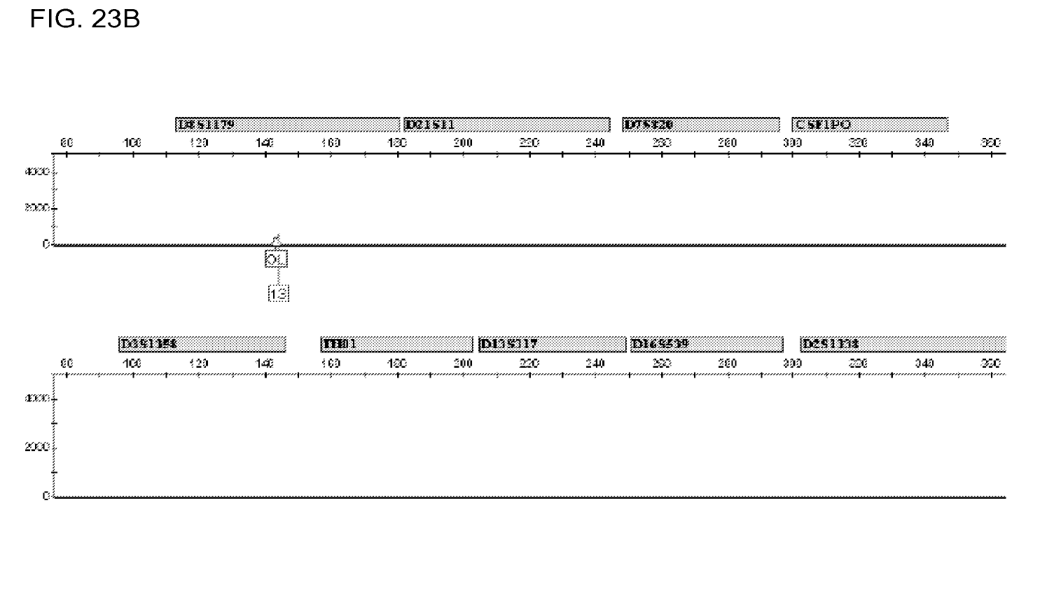

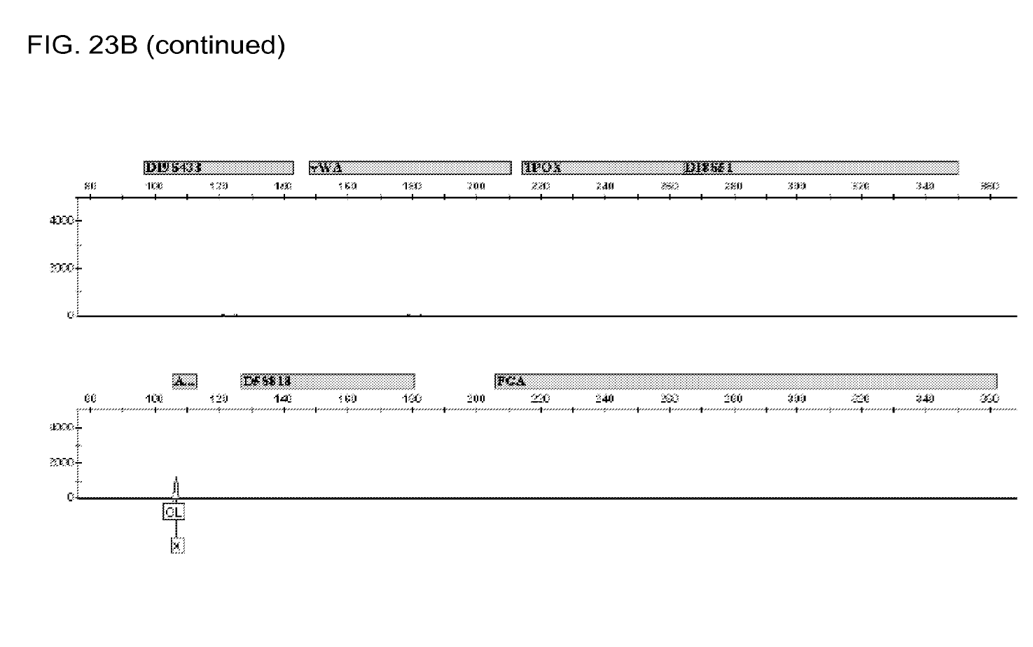

The following example shows the direct STR genotyping of crude human DNA samples containing tannins. The results show that the combination of the novel mutant enzyme and enhancer cocktail can tolerate the inhibition and generate a full genotyping profile, while a weak partial profile consisting of two peaks was produced with the ID_PLUS kit (see e.g., FIG. 23A and FIG. 23B).

### REFERENCES

Al-Soud W A, Ouis I S, Li D Q, Ljungh S, Wadström T. (2005) Characterization of the PCR inhibitory effect of bile to optimize real-time PCR detection of Helicobacter species. FEMS Immunol Med. Microbiol. 44(2):177-82.
Al-Soud W A and Rådström P. (1998) Capacity of Nine Thermostable DNA Polymerases To Mediate DNA Amplification in the Presence of PCR-Inhibiting Samples. Appl Environ Microbiol. 64(10): 3748-3753.
Andrews W H and Hammack T. (2007) Chapter 5: Salmonella. U.S. Food and Drug Administration (FDA) Bacteriological Analytical Manual (BAM) Online. http://www.cfsan.fda.gov/˜ebam.
Barnes W M. (1992) The fidelity of Taq polymerase catalyzing PCR is improved by an N-terminal deletion. Gene, 112, 29-35.
Barnes W M. (1994) Tips and tricks for long and accurate PCR, TIBS 19:342.
Barnes W M. (1994) PCR amplification of up to 35 kb DNA with high fidelity and high yield from bacteriophage templates, PNAS. 91:2216-2220.
Bickley J, Short J K, McDowell D G, Parkes H C. (1996) Polymerase chain reaction (PCR) detection of Listeria monocytogenes in diluted milk and reversal of PCR inhibition caused by calcium ions. Lett. Appl. Microbiol. 22:153-158.
Busta F F and Speck M L. (1968) Antimicrobial effect of cocoa on salmonellae. Appl Microbiol. 16(2): 424-425.
Cheng C M, Lin W, Van K T, Phan L, Tran N N, Farmer D. (2008). Rapid detection of Salmonella in foods using real-time PCR. J Food Prot.; 71(12):2436-41.
Downes F P and Ito K, eds. Compendium of Methods for the Microbiological Examination of Foods. Edition: 4, revised. American Public Health Association, 2001.
Feng P. (1997). Impact of Molecular Biology on the Detection of Foodborne Pathogens. Mol. Biotech. 7:267-278.
Feng P. (2001) Appendix 1, Rapid Methods for Detecting Foodborne Pathogens, U.S. Food and Drug Administration (FDA) Bacteriological Analytical Manual (BAM) Online. http://www.cfsan.fda.gov/˜ebam
Hedberg C. (1999) Food-related illness and death in the United States. Emerg Infect Dis. 5(6):840-2.
Isonhood J, Drake M, Jaykus L A. 2006 Upstream sample processing facilitates PCR detection of Listeria monocytogenes in mayonnaise-based ready-to-eat (RTE) salads. Food Microbiology. 23(6): 584-590.
Josefsen M H, Krause M, Hansen F, Hoorfar J. 2007. Optimization of a 12-hour TaqMan PCR-based method for detection of salmonella bacteria in meat. Applied and Environmental Microbiology, 73(9): 3040-3048.
Kermekchiev M B, Kirilova L I, Vail E E, Barnes W M. (2009) Mutants of Taq DNA polymerase resistant to PCR inhibitors allow DNA amplification from whole blood and crude soil samples. Nucleic Acids Res. 37(5):e40.
Knutsson R, Fontanesi M, Grage H, Rådström P. (2002) Development of a PCR-compatible enrichment medium for Yersinia enterocolitica: amplification precision and dynamic detection range during cultivation. Int. J. Food Microbiol. 72, 185-201.
Lantz P G, Tjerneld F, Borch E, Hahn-Hägerdal B, Rådström P. (1994) Enhanced sensitivity in PCR detection of Listeria monocytogenes in soft cheese through use of an aqueous two-phase system as a sample preparation method. Appl. Environ. Microbiol. 60:3416-3418.
Liu D. (2008) Preparation of Listeria monocytogenes specimens for molecular detection and identification. Int J Food Microbiol 122(3): 229-242.
Makino S, Okada Y, Maruyama T. (1995) A new method for direct detection of Listeria monocytogenes from foods by PCR. Applied and Environmental Microbiology 61, 3745-3747.
Ochman, H. and Groisman, E. (1996) Distribution of Pathogenicity Islands in Salmonella spp. Infection and Immunity, 64(12): 5410-5412.
Patel J R and Bhagwat A A. 2008. Rapid real-time PCR assay for detecting Salmonella in raw and ready-to-eat meats. Acta Vet Hung. 56(4):451-8.
Poelma P L, Andrews W H, Wilson C R. (1981) Pre-enrichment broths for recovery of Salmonella from milk chocolate and edible casein: collaborative study. J Assoc Off Anal Chem. 64(4):893-8.
Powell H A, Gooding C M, Garrett S D, Lund B M, Mckee R A. (1994) Proteinase inhibition of the detection of Listeria monocytogenes in milk using the polymerase chain reaction. Lett Appl Microbiol. 18:59-61.
Rådström P, Knutsson R, Wolffs P, Lövenklev M, Löfström C. (2004) Pre-PCR processing: strategies to generate PCR-compatible samples. Mol. Biotechnol. 26(2):133-46.
Rossen L, Nørskov P, Holmstrøm K, Rasmussen O F. (1992) Inhibition of PCR by components of food samples, microbial diagnostic assays and DNA-extraction solutions. Int J Food Microbiol. 17(1):37-45.
Shinozuka K, Kikuchi Y, Nishino C, Mori A, Tawata S. (1988) Inhibitory effect of flavonoids on DNA-dependent DNA and RNA polymerases, Cellular and Molecular Life Sciences (CMLS), 44: 882-885.
Wernars K, Heuvelman C J, Chakraborty T, Notermans S H. (1991) Use of the polymerase chain reaction for direct detection of Listeria monocytogenes in soft cheese. J. Appl. Bacteriol. 70:121-126.
Weschler, T. (2008) Food Micro-2008 to 2013, Strategic Consulting, Inc., Oct. 7, 2008.
Wilson I G. (1997) Inhibition and facilitation of nucleic acid amplification. Appl Environ Microbiol. 63:3741-3751.
Zapatka F A, Varney G W, Sinskey A J. (1972) Neutralization of the bactericidal effect of cocoa powder on salmonellae by casein. J Appl Bacteriol. 42(1):21-5.
Zhang Z, Kermekchiev M B, Barnes W M. Direct PCR Amplification of DNA from Crude Samples Using a PCR Enhancer Cocktail and Novel Mutants of Taq. J Mol Diagn Diagnostics (submitted, April, 2009).

1.-40. (canceled)

41. A method of amplifying a target nucleic acid with a polymerase chain reaction (PCR) in a sample comprising a PCR inhibitory substance, the method comprising:
(i) forming an assay mixture comprising
a sample comprising a target nucleic acid;
a PCR inhibitory substance;
at least one polymerase; and
an enhancer composition comprising at least one of (i) casein or (ii) polyvinylpyrrolidone (PVP) or a modified polymer of PVP, the enhancer composition present in an amount effective to reduce or eliminate inhibitory effects of the inhibitory substance in the sample; and
(ii) amplifying the target nucleic acid in the assay mixture.

42. The method of claim 41, wherein the inhibitory substance comprises a polyphenol.

43. The method of claim 41, wherein the enhancer composition comprises a casein selected from the group consisting of an αS1 casein, an αS2 casein, a β casein, a κ casein, or a paracasein.

44. The method of claim 41, wherein the enhancer composition comprises casein at a concentration of at least about 0.05%; at least about 0.05% up to about 2.5%; or about 0.4%.

45. The method of claim 41, wherein the enhancer composition comprises PVP or PVPP at a concentration of at least about 0.1%; at least about 0.1% up to about 25%; about 8% to about 10%; or about 1% to about 5%.

46. The method of claim 41, wherein the enhancer composition comprises at least one of trehalose, carnitine, a nonionic detergent, or heparin.

47. The method of claim 41, wherein the enhancer composition comprises about 0.1 to about 0.8 M trehalose, about 0.1 M to about 1.5 M L-carnitine, or about 0.01 to about 8% nonionic detergent.

48. The method of claim 41, wherein
the sample is an enriched sample; or
the sample is an enriched sample; and
the enhancer composition comprises:
(i) casein or PVP, or a modification thereof, and
(ii) (a) at least one of trehalose, carnitine, or a nonionic detergent; or (b) about 0.1 to about 0.8 M trehalose, about 0.1 M to about 1.5 M L-carnitine, and about 0.01 to about 8% nonionic detergent.

49. The method of claim 41, wherein the sample comprises:
(i) a plant material, blood or blood component, bile, dye, or soil; or
(ii) chocolate, potato skins, tea, berries, beer, wine, olive oil, walnuts, peanuts, tobacco, tomato, soybean, indigo dye, bile tannin, whole blood, blood serum, blood plasma, or soil.

50. The method of claim 41, wherein the sample comprises:
(i) chocolate at a concentration of about 0.05 μg/μl up to about 20 μg/μl;
(ii) soil or soil extract at a concentration of at least about 1% up to about 90% of a total volume of the assay mixture or a soil or soil extract equivalent amount that provides up to about 25 ng of humic acid per 50 μL reaction volume;
(iii) a plant material or plant extract at a concentration of at least about 1% up to about 90% of a total volume of the assay mixture or a plant material or plant extract equivalent amount that provides up to about 300 ng of polyphenols per 25 μl reaction volume;
(iv) blood or a blood component at a concentration of at least about 1% up to about 25% of a total volume of the assay mixture.

51. The method of claim 41 wherein the target nucleic acid comprises:
(i) a DNA or an RNA molecule;
(ii) a pathogen DNA or an RNA molecule; or
(iii) a Salmonella DNA or an RNA molecule.

52. The method of claim 41 wherein:
the PCR is a reverse-transcriptase (RT) PCR or a real-time RT-PCR;
the target nucleic acid comprises an RNA molecule; and
the assay mixture further comprises a reverse-transcriptase.

53. The method of claim 41, wherein the assay mixture comprises:
(i) at least one dye; or
(ii) at least one dye selected from the group consisting of SYBR Green, Ethidium Bromide, PICO, TOTO, YOYO or LC Green.

54. The method of claim 41, wherein the assay mixture comprises at least one dye present in the assay mixture at least about 0.5× up to about 50×, up to about 40×, up to about 30×, up to about 20×, or up to about 10×, where X is a manufacturer unit for concentration.

55. The method of claim 41, wherein the assay mixture comprises at least one DNA polymerase is selected from the group consisting of OmniTaq, Omni Klentaq, Omni Klentaq-LA, wild type Taq; FastStart Taq; JumpStart Taq; HotStart Plus Taq; AmpliTaq Gold, KlenTaq, FL-12, FL-10, and KT-12.

56. A composition for enhancing nucleic acid amplification in a sample comprising a PCR inhibitory substance, the method comprising:
(i) (a) casein at a concentration of at least about 0.05% up to about 2.5%; or (b) PVP or PVPP at a concentration of at least about 0.1% up to about 25%; and
(ii) about 0.1 to about 0.8 M trehalose, about 0.1 M to about 1.5 M L-carnitine, and about 0.01 to about 8% nonionic detergent.

57. The composition of claim 56 wherein the sample comprises polyphenols in an amount effective to inhibit PCR.

58. A method of culturing a microorganism comprising:
culturing a microorganism in a culture media, the culture media comprising PVP, or a modification thereof, or casein;
wherein the PVP or casein is present in an amount effective to decrease, substantially eliminate, or eliminate growth inhibition effects of a substance in the culture media or increase or substantially increase microorganism growth rate.

59. The method of claim 58, wherein the culture media comprises:
(i) a solid culture media, the solid culture media comprising PVP, or a modification thereof, in an amount of at least about 0% up to about 25% or casein in an amount of 0% up to 10%; or
(ii) a liquid culture media, the liquid culture media comprising PVP, or a modification thereof, in an amount of at least about 0% up to about 4% or casein in an amount of at least about 0% up to 10%.

60. The method of claim 58, wherein a concentration of PVP, or a modification thereof, in the culture media of greater than 4% inhibits microorganism growth.
